SEQUENCE LISTING (2 sequences)

SEQ ID NO: 1 - DNA, 185 bp, Homo sapiens
cagctgacattttttgtttgctctagaatgaacggtggaaggcggcaggccgaggcttttccgcccgctgaaagtcagcgagaaaaacagcgcgcggggagcaaaagcacggcgcctacgcccttctcagttagggttagacaaaaaatggccaccacccctcccaggcccaccctccgcaaccc

SEQ ID NO: 2 - DNA, 185 bp, Homo sapiens
gggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctg

(19) United States
(12) Patent Application Publication
(10) Pub. No.: US 2005/0282893 A1
(43) Pub. Date: Dec. 22, 2005

(54) Methods and compositions for using suramin, pentosan, polysulfate, telomerase antisense and telomerase inhibitors

(76) Inventors: Jessie L.-S. Au, Columbus, OH (US); M. Guillaume Wientjes, Columbus, OH (US)

Correspondence Address: Jerry K. Mueller, Jr.;Mueller and Smith, LPA, 7700 Rivers Edge Drive, Columbus, OH, 43235, US

(21) Appl. No.: 11/193,883

(22) Filed: Jul. 29, 2005

(30) Foreign Application Priority Data
Jan. 30, 2004 (WO) PCT/US04/02609

(51) Int. Cl. A61K 31/185; C12Q 1/68

(52) U.S. Cl. 514/553; 435/6

(57) ABSTRACT

The invention provides methods and compositions for inhibiting telomerase activity and treatment of telomerase mediated conditions or diseases. The methods, compounds, and compositions of the invention may be employed alone, or in combination with other pharmacologically active agents, surgery, or radiation in the treatment of conditions or diseases mediated by telomerase activity, such as in the treatment of cancer.

### CROSS-REFERENCE TO RELATED APPLICATIONS

This application claims benefit of International Application number PCT/US2004/002609, filed Jan. 30, 2004. This application also is cross-referenced to U.S. patent application Ser. No. 10/464,018, entitled “Methods and Compositions for Modulating Drug Activity through Telomere Damage”, filed on Jun. 18, 2003, and U.S. patent application Ser. No. 09/587,559, entitled “Methods and Compositions for Modulating Cell Proliferation and Cell Death”, filed on Jun. 5, 2000, the entire disclosure of which are incorporated herein by reference.

### GOVERNMENT SPONSORED RESEARCH

The work described in this application was supported, in part, by grants from the United States Department of Health and Human Services (Grant numbers R01CA77091, R01CA97067, R37CA49816; RO1CA78577; RO1 CA74179; R21CA91547; and U01CA76576).

### BACKGROUND OF THE INVENTION

1. Field of the Invention

The present invention describes an antisense molecule that targets the RNA portion of human telomerase (hTR-antisense) and inhibits telomerase activity in human cancer cells. The invention also describes the use of suramin, pentosan polysulfate (PPS), or hTR-antisense to inhibit telomerase activity. The invention further teaches the use of another telomerase inhibitor, 3′-azido-deoxythymidine (AZT), at nontoxic doses producing plasma concentrations in the nanomolar range, to treat tumors. The present invention further teaches a method of treating a tumor by using a telomerase inhibitor concurrently with or after other means of surgical or non-surgical tumor size reduction (cytoreduction). The invention further teaches the use of a telomerase inhibitor prior to non-surgical tumor size reduction treatments, to sensitize tumor cells to the effects of such treatments. The invention further teaches the use of a telomerase inhibitor, e.g., suramin, PPS, or hTR-antisense, to prevent the development of cancer.

2. Description of the Prior Art

Telomere and telomerase. Telomeres are the structures capping the ends of chromosomes, and are critical to the maintenance of chromosomal integrity and replication potential. Due to the inability to replicate the 3′ end of chromosomes by DNA polymerases, telomeres are shortened by 50 to 200 bp per cell division. Loss of telomeres to below a critical minimum length results in cell death (Lingner, et al., Science, 269:1533-1534, 1995), or causes cells to enter senescence (e.g., loss of proliferative capacity) (Goldstein, Science, 249:1129-1133, 1990; Martin, et al., Lab. Invest., 23:86-92, 1979). The enzyme telomerase, consisting of RNA and protein components, is capable of restoring telomere length, and is nearly universally present in tumor cells and usually absent in normal somatic cells (Hiyama, et al., J Natl Cancer Inst, 88:116-122, 1996).

Methods to use telomerase inhibitors. The critical nature of the function and the selective presence of telomerase in tumor cells led to the initial hypothesis that telomerase is a desirable tumor-specific target and that telomerase inhibitors are useful therapeutic anticancer agents (e.g., in Huminiecki, L., Acta Biochimica Polonica, 43:531-538, 1996). This proposed use was based on the hypothesis that active telomerase is needed for initiating and maintaining neoplasia. No evidence of clinical effectiveness of telomerase inhibition has been reported. In fact, several important findings have since been described; all of which indicate that telomerase inhibitors are of limited therapeutic value. First, it now is recognized that there are other telomere lengthening mechanisms that are independent of telomerase (e.g., Gan, et al., FEBS Lett., 527:10-14, 2002). Second, the pre-existing telomeres in tumor cells are usually of sufficient length to support multiple rounds of cell proliferation, even when telomerase is completely inhibited. Accordingly, telomerase inhibitors do not cause cytotoxicity until after a significant lag time. For example, telomerase inhibitors resulted in cytotoxicity in HeLa cells after 23 to 26 cell doublings (Feng, et al., Science, 269:1236-1241, 1995). Because uncontrolled tumor growth will lead to lethal tumor burden, e.g., tumor-mediated death in a patient usually occurs after less than 10 doubling of the tumor size, the requirement of the long lag time for telomerase inhibition to cause cell death or senescence render telomerase inhibitors impractical and not useful therapeutic agents. To date, no drugs specifically identified as telomerase inhibitors have been tested as anticancer agents in human patients, even though the hypothesis of using telomerase inhibitors to treat cancer first appeared nearly 10 years ago (U.S. Pat. No. 5,489,508).

In U.S. patent application Ser. No. 10/464,018, Applicants describe the discovery that treatment of a cancer with paclitaxel causes telomere damage thereby inducing telomerase activity and leading to resistance to paclitaxel treatment in the cancer, and the discovery that co-administration of a telomerase inhibitor (e.g., AZT, hTR-antisense) diminishes this resistance and enhances the anti-tumor effect of paclitaxel.

The present application extends Applicants' earlier invention and teaches methods to make telomerase inhibition an effective antitumor treatment. In one aspect, the invention teaches using a cytoreductive treatment, administered prior to or concurrent with the administration of a telomerase inhibitor, to reduce the tumor burden, so that the tumor burden would not reach the lethal level before the telomerase inhibitor can erode the telomere length to below the critical level for apoptosis and cell senescence to occur and, thereby, allow telomerase inhibition to become an effective treatment. Hence, the invention teaches methods to use telomerase inhibitors to combat cancer, comprising of administering to the cancer patient a cytoreductive treatment (i.e., treatment that reduces the tumor size, e.g., surgery, radiation therapy, chemotherapy, etc.) followed by administering a telomerase inhibitor, wherein the plasma concentrations of the telomerase inhibitor are maintained at telomerase-inhibitory levels for a long duration, e.g., several weeks or several months. The telomerase inhibitor also may be given concurrently with the cytoreductive treatment. This teaching is based on Applicants' discovery that maintenance of suramin in plasma at telomerase-inhibitory, but nontoxic, concentrations during and/or after completion of a cytoreduction treatment, e.g., chemotherapy, enhanced tumor shrinkage, retarded tumor progression and extended the survival time in human cancer patients.

Another aspect of the current invention teaches the use of a telomerase inhibitor after terminating a cytotoxic treatment, e.g., due to the dose-limiting toxicity of the cytotoxic treatment. Cytotoxic treatments, such as chemotherapy, usually cause toxicity in cancer patients and, therefore, cannot be given indefinitely (i.e., as a maintenance treatment for a maintenance time period). This is an important problem from the clinical standpoint, as most cancers treated by chemotherapy are advanced and typically cannot be eradicated. For example, less than 1% of nonsmall cell lung cancer patients achieve complete response where the tumors are eradicated by chemotherapy (Schiller, et al., New Eng J Med, 346:92-98, 2002). The residual tumor cells, therefore, can resume growth after the cessation of cytotoxic treatments. In contrast, telomerase inhibitors, due to the selective expression of telomerase in tumor cells, are not likely to cause toxicity and, therefore, can be given indefinitely or over long period of time. The ability of the telomerase inhibitors to inhibit cell proliferation or induce cell senescence, in turn, will prevent tumor regrowth and lead to survival advantage.

For example, a common and effective chemotherapy in advanced nonsmall cell lung cancer human patients is the combination of paclitaxel (225 mg/m²) and carboplatin (AUC=6) given every 3 weeks. The neurotoxicity of paclitaxel is cumulative and becomes dose-limiting usually after 4 to 6 treatments, necessitating the termination of therapy. The tumor typically starts to resume growth within several months, resulting in average survival time of about 8 months (Schiller, et al., 2002).

With respect to prior art in this area, WO 97 38013 A teaches the use of peptide nucleic acid antisense molecules to telomerase (PNA) and antineoplastic agents in combination. Kondo described that the inhibition of telomerase increases the susceptibility of tumors to DNA damaging drugs (Kondo, et al., Oncogene, 16:3323-3330, 1998). Hence, these previous publications teach the generally accepted concept that a combined treatment of a cancer with two effective modalities may be more effective than treatment with each of the separate modalities. However, these publications did not disclose the use of a cytoreductive treatment for the purpose of reducing the tumor size sufficiently to make treatment with a telomerase inhibitor effective. These publications also do not disclose the use of telomerase inhibitors after terminating a cytotoxic treatment as a means to combat cancer. Hence, Applicants' discovery that a cytoreductive treatment is necessary to render telomerase inhibition as an effective anticancer treatment is novel as no such use or results have been previously described. Applicants' discovery that low doses of suramin that maintain plasma suramin concentrations sufficient to inhibit telomerase, but insufficient to cause antitumor activity when given together with a cytoreductive treatment, were effective against cancer in human patients also is surprising as the prior art shows that such low doses of suramin have no antitumor activity in humans (Dreicer, et al., Invest. New Drugs, 17:183-189, 1999; Falcone, et al., Tumori, 84:666-668, 1998; Falcone, et al., Cancer, 86:470-476, 1999; Hussain, et al., J of Clin. Oncol., 18:1043-1049, 2000; Miglietta, et al., J Cancer Res. Clin. Oncol., 123:407-410, 1997; Mirza, et al., Acta Oncologica, 36:171-174, 1997; Motzer, et al., Cancer, 72:3313-3317, 1993; Rapoport, et al., Ann. Oncol., 4:567-573, 1993; Rosen, et al., J. Clin. Oncol., 14:1626-1636, 1996).

The present invention also teaches that pretreatment with a telomerase inhibitor enhances the antitumor effect of chemotherapy. This is based on Applicants' discovery that pretreatment with low doses of suramin that yield plasma concentrations of e.g., less than 50 micromolar, which were sufficient to inhibit telomerase, but insufficient to cause cytotoxicity, enhanced the activity of chemotherapy. The chemosensitization effect of suramin was observed when low doses of suramin were administered before or after tumor establishment, or when the tumor burden was not life-threatening. This discovery is novel as no such use or results have been previously described. In fact, this discovery is surprising as the prior art shows that such low doses of suramin have no antitumor activity before or after tumor establishment, in nonhuman animals (e.g., Pesenti, et al., Br. J. Cancer, 66:367-372,1992).

Telomerase inhibitors. The current application teaches the use of suramin, PPS, or hTR-antisense to inhibit telomerase. This is based on Applicants' discovery that suramin, PPS, and hTR-antisense are effective telomerase inhibitors and reduce the telomere length in tumors implanted in animals. The prior art teaches several compounds with telomerase-inhibitory properties (e.g., those described in U.S. Pat. Nos. 5,656,638, 5,760,062, 5,767,278, 5,770,613, and 5,863,936). The prior art also teaches that cisplatin inhibits telomerase inhibition, possibly due to crosslinking of the telomeric repeat sequences (Burger, et al., Eur J Cancer, 33:638-644, 1997). The prior art further teaches the use of peptide nucleic acid (PNA) antisense molecules and phosphorothioate oligonucleotides to inhibit telomerase activity by targeting the RNA component of telomerase (Norton, et al., Nat Biotechnol, 14:615-619, 1996). However, none of these reports teaches the use of suramin, PPS, or hTR-antisense as telomerase inhibitors.

Telomerase inhibitors as chemopreventives. Chemoprevention using telomerase inhibitors has been proposed. For example, Akama indicated the possibility of administering the telomerase inhibitors of the class of thiazolidinone compounds as a prophylactic (Akama, et al., U.S. Pat. No. 6,452,014, 2002). However, Akama does not teach using suramin, PPS, or hTR antisense to inhibit telomerase.

Methods to use suramin. Suramin, a polysulfonated naphthylurea, has multiple pharmacological activities (Ahmann, et al., Proc. Am. Soc. Clin. Oncol., 10:178, 1991; Armand, Bonnay, Gandia, Cvitkovic, De Braud , Bertheault, Droz, Carde, Schlumberger, and Fourcault, 1991; Dreicer, et al., 1999; Falcone, et al., 1998; Falcone, et al., 1999; Garrett, et al., Proc. Natl. Acad. Sci. USA, 81:7466-7470, 1984; Grazioli, et al., Int J Immunopharmacol, 14:637-642, 1992; Hawking, Adv. Pharmacol. Chemother., 5:289-322, 1978; Hensey, et al., FASEB, 258:156-158, 1989; Hosang, J. Cell. Biochem., 29:265-273, 1985; Hussain, et al., 2000; Manetti, et al., Bioorganic & Med. Chem., 6:947-958, 1998; Mills, et al., Cancer Res., 50:3036-3042, 1990; Myers, et al., Proc. Am. Soc. Clin. Oncol., 9:113, 1990; Ono, et al., Eur. J. Biochem., 172:349-353, 1998; Pollak, et al., Proc. Am. Soc. Clin. Oncol., 9:54, 1990; Pollak, et al., J. Natl. Cancer Inst., 82:1349-1352, 1990; Stein, Cancer Res., 54:2239-2248, 1993; Takano, et al., Cancer Res., 54:2654-2660, 1994; Wade, et al., J. Surg. Res., 53:195-198, 1992; Waltenberger, et al., J. Mol. Cell Cardiol., 28:1523-1529, 1996). Its antitumor activity is believed to be due to inhibition of DNA polymerase a and reverse transcriptase, inhibition of binding of growth factors (i.e., platelet-derived growth factor, fibroblast growth factors or FGF, transforming growth factor-β, epidermal growth factor, vascular endothelial growth factor, and insulin-like growth factor-1) and binding of IL-2 and transferrin to their respective receptors, inhibition of phosphorylation activity of PKC, and glycosaminoglycan metabolism. Suramin also inhibits Na/K-ATPase, tumor necrosis factor α, and topoisomerase II. Inhibition of telomerase activity by suramin, at any concentration, is not known, and was a novel finding by the Applicants.

Suramin has shown some activity in prostate cancer (DeClercq, Cancer Letters, 8:9-22, 1979; Eisenberger, et al., Cancer Treat Rev., 20:259-273, 1994; Fotes, et al., Biochim Biophys Acta., 38:262-272, 1973; Huang, et al., Oncogene, 9:491-499, 1994) and has been tested in a wide variety of solid tumors, either as single agent or in combination with other chemotherapeutics. The therapeutic plasma concentration of suramin is between 100 to 200 μM (140-280 μg/ml) (Funayama, et al., Anticancer Res., 13:1981-1988, 1993), with 70 to 210 μM (100-300 μg/ml) indicated as widest limits (Klohs, et al., U.S. Pat. No. 5,597,830, 1997). At these concentrations, suramin shows significant toxicities and only modest activity in patients. Multiple groups of investigators have shown that single agent therapy using suramin has no appreciable antitumor effects in human patients and that a combination of high dose suramin and a cytotoxic agent does not produce beneficial results in human patients as compared to single agents. Based on their findings, these same investigators recommend against using suramin, either as a single agent or in combination with other cytotoxic agents (Dreicer, et al., 1999; Falcone, et al., 1998; Falcone, et al., 1999; Hussain, et al., 2000; Miglietta, et al., 1997; Mirza, et al., 1997; Motzer, et al., 1993; Rapoport, et al., 1993; Rosen, et al., 1996). Hence, absent the discoveries upon which the present invention is based, there is no motivation to use suramin, either a single agent, or as combination of suramin in any dose with a cytotoxic agent, or to use the combination of subtherapeutic and nontoxic doses of suramin with a cytotoxic agent. The only exception would be the use of suramin at subtherapeutic doses, to enhance the action of other treatment modalities. This use, discovered by Applicants, and described in detail in U.S. Pat. No. 6,599,912, requires the administration of suramin shortly before, during, and shortly after, treatment of the patient with other modalities, such as chemotherapy or radiation. The current invention supports additional and different uses of suramin, and emphasizes the need for long total treatment duration, e.g., weeks, months, years, or indefinite time, so as to inhibits telomerase and cause sufficient telomere shortening to inhibit cell proliferation or cause cell death.

The present invention teaches the requirement of continuous inhibition of telomerase. Hence, a compound with a slow elimination from the body is desired. Suramin fulfills such requirement. The pharmacokinetics of suramin in human patients is characterized by a triphasic plasma concentration decline, with half-lives of 5.5 hours, 4.1 days, and 78 days. The total body clearance is 0.0095 liter/hour/m² (Jodrell, et al., J Clin. Oncol., 12:166-175, 1994). The disposition of suramin in dogs is also slow with a terminal half-life of about 13 days (See Example 8).

Methods to use PPS. PPS is a semi-synthetic heparinoid and has anti-coagulant effect (Wellstein, et al., Breast Cancer Res. Treat., 38:109-119, 1996). At concentrations that had no anticoagulation effect in patient sera and that were 1,000-fold lower than its cytotoxic effects, PPS inhibits the binding of FGF to their receptors and also inhibits angiogenesis in the chicken chorioallantoic membrane assay (Parker, et al., J. Natl. Cancer Inst., 85:1068-1073, 1993; Wellstein, et al., 1996). Anticoagulation is only found at concentrations above 1 μg/ml (Parker, et al., 1993). Under in vivo conditions, PPS inhibits the growth of the rat MAT-LyLu tumor, if treatment is started when the tumor is not palpable, but has little effect against established tumors and cannot inhibit the metastasis of MCF7 tumor in mice (McLeskey, et al., Br. J. Cancer, 73:1053-1062, 1996; Nguyen, et al., Anticancer Res., 13:2143-2148, 1993; Wellstein, et al., J. Natl. Cancer Inst., 83:716-720, 1991). The antitumor activity of PPS in preclinical tumor models has led to its clinical evaluation. The results show that PPS was well tolerated when the dose was adjusted to avoid its anti-coagulant effect, but did not show antitumor activity in patients (Lush, et al., Ann Oncol., 7:939-944, 1996; Swain, et al., Invest. New Drugs, 13:55-62, 1995). As a result, PPS is no longer being evaluated as a potentially useful antitumor or antiangiogenic agent. A search of the PDQ Clinical Trial Database (http://www.nci.nih.gov) for all active cancer clinical trials, as of Jan. 27, 2000, shows 1,790 trials worldwide, but no trials on PPS. Hence, absent the present discovery, there are no motivations to use PPS to treat cancer.

The doses of PPS used for antitumor activity evaluation were about 400 mg per meter squared per day, equivalent to about 10 mg/kg per day, and were given orally. Plasma concentrations increased over time of continuous daily administration, reaching 200-460 ng/ml on the fifteenth day of treatment. At this dose level, hematologic toxicities such as diarrhea, gastrointestinal bleeding, or proctitis, usually occurred within 1 to 3 month. Proctitis was the dose limiting toxicity in this trial (Marshall et al., Clin. Cancer Res., 3:2347-2354, 1997). The PPS concentrations required for 50% inhibition of telomerase activity (0.5-0.6 μg/ml, see Example 3) were substantially higher than the concentrations that cause proctitis toxicity. This consideration, together with the discovery that local administration of suramin to the targeted organ resulted in telomerase-inhibitory concentrations in the targeted tissues (e.g., 5 to 100 μg/g) but very low concentrations of suramin in the plasma (e.g., 0.1 μg/ml, see Example 13), led to the invention of local administration of PPS to the organs where telomerase inhibition is desired. This new use eliminates the potential problem of systemic host toxicities.

Methods to use AZT. Melana et al., recently summarized the history on AZT (Melana, et al., 1998). AZT is used to treat patients infected with the human immunodeficiency virus. AZT originally was developed as an antitumor agent. However, it is no longer considered a potential antitumor agent because of its relatively high cytotoxicity in animals when administered in drinking water. AZT was later found to have very low toxicity when it was administered as a bolus injection. AZT has since been tested in Phase I and II clinical trials, either as single agent or in combination with other drugs, in the treatment of gastrointestinal cancers. All of these earlier studies used doses of AZT (i.e., 7 to 10 g/m²/day) that would produce plasma concentrations in excess of 10 micromolar, as calculated based on the available data, as follows. The mean steady-state concentration of AZT in patient plasma after the usual oral dose of 2.5 mg/kg every four hours, or 15 mg/kg/day, is 1.06 μg/ml (Physician's Desk Reference, 2003). For cytotoxic treatment with AZT, a minimal dose of 3 g/m²/day is used (U.S. Pat. No. 5,116,823), which converts to approximately 85 mg/kg/day. Linear extrapolation of the steady-state concentration from 1.06 μg/ml for 15 mg/kg/day yields an expected plasma concentration of 6 μg/ml, or 22 μM, for a dose of 85 mg/kg/day. The prior art teaches that AZT is a telomerase inhibitor; the concentrations that produces 50% inhibition ranged from micromolar to millimolar (Strahl, et al., Nucleic Acid Res, 22:893-900, 1994; Strahl, et al., Mol. Cell Biol., 16:53-65,1996).

The present invention teaches the use of low doses of AZT. Applicants discovered that administration of nontoxic doses of AZT that delivered nanomolar plasma concentrations resulted in elimination of well-established tumors in animals. Addition of such nontoxic doses of AZT also enhanced the antitumor activity of paclitaxel in animals. Although AZT has been evaluated as an antitumor agent and a telomerase inhibitor, no prior art describes the use or the effectiveness of AZT at such low doses. In fact, Applicants' finding of the antitumor activity of AZT occurring at low doses resulting in nanomolar concentrations is surprising in view of the prior art showing that antitumor activity of AZT is found at much higher doses that result in micromolar concentrations in the plasma (Clark, et al., J Cancer Res. Clin. Oncol., 122:554-558, 1996). The finding that AZT, at nanomolar concentrations, enhances the activity of chemotherapy is also surprising in view of the prior art showing that the cytotoxic or telomerase-inhibitory concentrations of AZT are in the micromolar or millimolar range (Strahl, et al., 1994; Strahl, et al., 1996; (Melana, et al., Clin. Cancer Res., 4:693-696,1998; Table 1 in Example 1).

Antisense to telomerase. Inhibition of telomerase activity by antisense constructs has been reported, and the possibility of their application in the treatment of cancer has been proposed (Kelland, Lancet Oncol, 2:95-102, 2001). However, the hTR antisense reported here has not been described. Further, combining long-term treatment of telomerase inhibition through the use of telomere antisense constructs, with cytoreductive treatments, have not been proposed or described.

### SUMMARY OF THE INVENTION

The current application is based on several related discoveries on telomerase inhibition and telomere shortening. First, Applicants discovered that suramin, PPS, and hTR-antisense are effective telomerase inhibitors and reduce the telomere length in tumors implanted in animals. The second discovery is that pretreatment with suramin enhances the activity of chemotherapy against well-established tumors in tumor-bearing animals. The third discovery is that telomerase inhibitors, such as suramin and PPS, enhanced the anticancer activity of cancer chemotherapy and radiation. The fourth discovery is that maintenance of suramin in plasma at telomerase-inhibitory concentrations during and after completion of a cytoreductive treatment promoted tumor shrinkage, delayed tumor growth and extended the survival time in human cancer patients. The fifth discovery is that local administration of suramin to a tissue that was the intended target for treatment resulted in local tissue concentrations that were sufficient to inhibit telomerase and shorten telomere in the tissue but at the same time resulted in very low suramin concentrations in the plasma that would not have been sufficient to inhibit telomerase. The sixth discovery was that administration of nontoxic doses of AZT that delivered nanomolar plasma concentrations resulted in elimination of well-established tumors in animals and enhanced the antitumor activity of paclitaxel in animals.

Hence, Applicants present methods, compounds, and compositions, which can be applied to the treatment of a wide variety of tumors, and to improve the treatment outcome compared to standard treatments.

In the first aspect, the present invention teaches methods of inhibiting telomerase by contacting telomerase or cells containing telomerase with suramin, PPS, or hTR-antisense.

A second and related aspect teaches methods of inhibiting telomerase activity in a patient, preferably a mammal, suffering from a telomerase-mediated condition or disease, comprising administration to the patient of a therapeutically effective amount of suramin, or another telomerase inhibitor.

A third aspect teaches methods of improving the treatment outcome of a cancer patient, preferably a mammal, comprising administration to the patient of a telomerase-inhibitory amount of suramin, or another telomerase inhibitor. The telomerase-inhibiting treatment is given in such a way that the tumor is exposed to a telomerase inhibitor for a duration of at least several cycles of proliferation and, more preferably, at least 10 or 20 cycles of proliferation.

A fourth aspect teaches methods of enhancing the treatment of a cancer patient, preferably a mammal, comprising administering to the patient a telomerase-inhibitory amount of suramin, or another telomerase inhibitor, during and after completion of a cytoreductive treatment. Such cytoreductive treatment can be surgical excision of tumors or non-surgical treatments, e.g., radiation therapy, chemotherapy, photodynamic therapy. One or more telomerase inhibitors may be used to obtain better treatment results.

In a fifth aspect, the invention teaches a method of treating a cancer in a patient by first identifying a patient about to have a cancer, or harboring a cancer that is too small to be detected, and then administering a telomerase inhibitory agent to the patient such that treatment of the cancer, or prevention of cancer development, is achieved. Treatment of such patients with a telomerase inhibitory agent would be effective, as the small initial size of the tumor would allow many tumor proliferation cycles before the tumor burden would threaten the health and well being of the patient.

In a sixth aspect, the invention is a method of treating a patient in remission, after a successful treatment of his or her cancer, but where the patient remains at a substantial risk of developing a new or recurrent cancer. This is especially so for an agent of minimal or no toxicity to the patient, as it provides for a favorable risk-to-benefit ratio. Suramin, PPS, or AZT, inter alia, at the low amounts needed to inhibit telomerase activity, are minimally or not toxic to the patient, and provide such favorable risk-to-benefit ratio.

A seventh aspect teaches methods of inhibiting telomerase by transfecting cells with the hTR-antisense.

In an eighth aspect, the invention teaches the use of nontoxic doses of AZT yielding plasma concentrations below the micromolar range, e.g., in the nanomolar range, to combat cancer.

In a ninth aspect, the telomerase inhibitor can be administered locally, near the site of a known tumor, or in an organ where the current or future occurrence of a tumor is suspected. The locally administered telomerase inhibitor will provide effective telomerase-inhibitory concentrations to tumor cells or tissue located in proximity to the administration site. Local administration, e.g., intraluminal or other injection or implantation, can be in the form of a depot, such as a slow release device. Local administration of the telomerase inhibitor is to provide telomerase-inhibitory concentrations in the tissues that are targets of the treatment, but does not need to result in telomerase-inhibitory concentrations in plasma or other organs that are not the targets of the treatment.

In a separate, cross-referenced patent application (U.S. patent application Ser. No. 09/587,559), Applicants described the discovery that acidic and basic fibroblast growth factors (FGF) cause broad-spectrum tumor resistance to chemotherapy and that inhibitors of FGF enhance the antitumor activity of cancer chemotherapy and radiation therapy. Suramin and PPS are FGF inhibitors. The current invention teaches that the same compounds inhibit telomerase activity. The concentrations of suramin and PPS that produce FGF inhibition also produce telomerase inhibition. Thus, suramin or PPS can be administered to a patient in combination with a cytotoxic anti-cancer treatment to enhance be effect of the cytotoxic agents, while at the same time inhibiting telomerase to decrease the telomere length and thereby achieve additional, beneficial antitumor effect. After completion of the cytotoxic treatment regimen, suramin or PPS administration can be maintained to achieve a long-term inhibition of telomerase activity. The use of the same agent(s) to target both the FGF-resistance and telomerase-resistance mechanisms represents a convenience to the patient and to the treating physician and improves the outcome of the cytotoxic treatment.

As explained above, effective anti-cancer treatment with a telomerase inhibitor requires inhibition of telomerase activity over multiple proliferation cycles of the tumor cells. Such continuous inhibition is most effectively achieved with an agent that has a long terminal half-life in the patient, for example longer than one week, so as to continuously maintain inhibition of telomerase activity with treatment at infrequent intervals. Suramin, with an elimination half-life in excess of one week in humans (Jordell et al., 1994) and nonhuman animals (see Example 8), fulfills this requirement.

The compounds of the invention have many valuable uses as inhibitors of deleterious telomerase activity, such as, for example, in the treatment of cancer in mammals, such as humans. The pharmaceutical compositions of the invention may be employed in treatment regimens in which cancer cells are killed, in vivo, or can be used to kill cancer cells ex vivo. Thus, this invention provides therapeutic compounds and compositions for treating cancer, and methods for treating cancer and other telomerase-mediated conditions or diseases in humans and other mammals (e.g., dogs, cats, cows, horses, and other animals of veterinary interest).

Other features and advantages of the invention will be apparent from the following detailed description, and from the claims.

### DETAILED DESCRIPTION OF THE INVENTION

In order to provide a clear and consistent understanding of the invention, certain terms employed in the specification, examples, and the claims are, for convenience, collected here.

Definitions

As used herein, the term “aberrant growth” refers to a cell phenotype, which differs from the normal phenotype of the cell, particularly those associated either directly or indirectly with a disease such as cancer.

As used herein, the term “administering” refers to the introduction of an agent to a cell, e.g., in vitro, a cell in an animal, i.e., in vivo, or a cell later placed back in the animal (i.e., ex vivo).

As used herein, the terms “agent”, “drug”, “compound”, “anticancer agent”, “chemotherapeutic”, “antineoplastic”, and “antitumor agent” are used interchangeably and refer to agent/s (unless further qualified) that have the property of inhibiting or reducing aberrant cell growth, e.g., a cancer. The foregoing terms are also intended to include cytotoxic, cytocidal, or cytostatic agents. The term “agent” includes small molecules, macromolecules (e.g., peptides, proteins, antibodies, or antibody fragments), and nucleic acids (e.g., gene therapy constructs, recombinant viruses, nucleic acid fragments (including, e.g., synthetic nucleic acid fragments).

As used herein, the term “apoptosis” refers to any non-necrotic, cell-regulated form of cell death, as defined by criteria well established in the art.

As used herein, the terms “benign”, “premalignant”, and “malignant” are to be given their art recognized meanings.

As used herein, the terms “cancer”, “tumor cell”, “tumor”, “leukemia”, or “leukemic cell” are used interchangeably and refer to any neoplasm (“new growth”), such as a carcinoma (derived from epithelial cells), adenocarcinoma (derived from glandular tissue), sarcoma (derived from connective tissue), lymphoma (derived from lymph tissue), or cancer of the blood (e.g., leukemia or erythroleukemia). The terms “cancer” and “tumor cell” also are intended to encompass cancerous tissue or a tumor mass, which shall be construed as a compilation of cancer cells or tumor cells. In addition, the terms “cancer” and “tumor cell” are intended to encompass cancers or cells that may be either benign, premalignant, or malignant. Typically a cancer or tumor cell exhibits various art recognized hallmarks such as, e.g., growth factor independence, lack of cell/cell contact growth inhibition, and/or abnormal karyotype. By contrast, a normal cell typically can only be passaged in culture for a finite number of passages and/or exhibits various art recognized hallmarks attributed to normal cells (e.g., growth factor dependence, contact inhibition, and/or a normal karyotype).

As used herein, the term “cell” includes any eukaryotic cell, such as somatic or germ line mammalian cells, or cell lines, e.g., HeLa cells (human), NIH3T3 cells (murine), embryonic stem cells, and cell types such as hematopoietic stem cells, myoblasts, hepatocytes, lymphocytes, and epithelial cells and, e.g., the cell lines described herein.

As used herein, the term “identifying a patient having or about to have” refers to a patient having been determined to have, or to be statistically likely to have, a cancer using various art recognized diagnostic or prognostic techniques including, e.g., the prostate specific antigen (PSA) test, BRCA1 and/or BRCA2 genotyping, genetic profiling, etc. The term is also intended to include the mere knowing or receipt of any information (e.g., a prognosis, diagnosis) indicating that the patient is having or about to have a cancer.

As used herein, the term “inhibiting or reducing the growth of a cell” e.g., a cancer cell, refers to the slowing, interrupting, or arresting of its growth and/or metastasis, and can, but does not necessarily require, e.g., a total elimination of the aberrant growth of the cell. The term is also intended to encompass inhibiting or reducing cell growth via cell death (apoptosis) or necrosis.

As used herein, the terms “locally” and “regionally” are used interchangeably, and refer to the administration of a therapy into a tumor mass, into a tumor-bearing organ, or in a general tumor field or area suspected to be seeded with metastases, or premalignant lesions, e.g. an organ specific for a tumor type such as prostate for prostate cancer.

As used herein, the term “tumor burden”, a term widely recognized in the art, refers to the partial or total mass, volume or size of tumor tissues in a patient. The tumor size is determined by standard clinical means, usually consisting of palpation, or imaging methods (e.g., X-ray, CAT scan, PET scan, ultrasound sonogram). The tumor burden can be estimated from a summation of the tumor sizes. The tumor burden is an art recognized indicator of the clinical course of the disease, where a large tumor burden indicates a bad prognosis, while a small tumor burden is a positive prognostic sign. Tumor burden is often regarded in relationship to the lethality of the tumor for the patient. While human patients can often tolerate a tumor burden of about 1 kg, this cannot be taken as a general rule, as the location of the tumor, and the damage that can be caused by the tumor are important determinants of the lethality associated with the tumor mass. For example, a metastatic tumor growing in the brain can be lethal at a size of a few centimeters, while much larger tumors in the liver or viscera can be tolerated. Hence, a lethal tumor burden for a patient with a tumor metastatic to the brain can be much smaller than a lethal tumor burden for a patient with no such metastases.

As used herein, the term “systemically” refers to the administration of a therapy with the intent that the agent will be widely disseminated throughout subject, such as by oral or intravenous administration. Similarly, “systemic” concentrations refer to concentrations throughout the body, such as found in circulating plasma.

As used herein, the term “pharmaceutically acceptable carrier” is art recognized and includes a pharmaceutically acceptable material, composition or vehicle, suitable for administering compounds of the present invention to mammals.

As used herein, the term “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient (e.g., a therapeutically-effective amount) in combination with a pharmaceutically acceptable carrier.

As used herein, the term “subject” is intended to include human and non-human animals (e.g., mice, rats, rabbits, cats, dogs, livestock, and primates). Preferred human animals include a human patient having a disorder characterized by aberrant cell growth, e.g., a cancer.

As used herein, the term “telomere” refers to the end of a eukaryotic chromosome, which is frequently abnormally extended in a cancer cell.

As used herein, the term “telomerase” refers to the cellular enzyme or enzyme activity directed to the nucleotide polymerization or maintenance of chromosome ends known as telomeres.

As used herein, the terms “telomerase inhibitory agent” and “telomerase inhibitor” refer to an agent that inhibits (completely or partially) the activity of the enzyme telomerase.

As used herein, the term “inhibition of telomerase” refers to a directly measurable inhibition of the telomerase enzyme, for example, as demonstrated by using the modified TRAP assay described by Gan et al., (Gan et al., Pharm. Res., 18:488-493, 2001), or based on the reduction of the average telomere length in all of the cells as demonstrated by using the TALA assay described by Gan et al., (Gan et al., Pharm. Res., 18:1655-1659, 2001) or erosion of individual telomeres in individual cells using the FISH assay described by Gan et al., (Gan et al., Pharm. Res., 18:1655-1659, 2001).

As used herein, the term “chemosensitizer” refers to an agent that increases the antitumor effect of a second agent, e.g., an anticancer chemotherapeutic agent. The term “chemosensitization” refers to an increase in antitumor activity of a cancer chemotherapeutic agent by a chemosensitizer when compared to the antitumor activity of the cancer chemotherapeutic agent given without the chemosensitizer.

As used herein, the term “chemoprevention” refers to using an agent to prevent the development of a tumor, a cancer. The term “chemopreventive” refers to an agent that prevents the development of a tumor, a cancer.

As used herein, the term “therapeutically-effective amount” of a telomerase inhibitory agent and/or chemotherapeutic refers to the amount of such an agent which, alone or in combination, is effective, upon single- or multiple-dose administration to the subject, e.g., a human patient, at inhibiting telomerase activity (for a telomerase inhibitory agent), or at inhibiting or reducing aberrant cell growth, e.g., a cancer (for a chemotherapeutic).

As used herein, the term “pentosan polysulfate” or “PPS” refers to a semi-synthetic sulfated polyanion composed of beta-D-xylopyranose residues with properties similar to heparin, with molecular weight ranges from 1500-5000. The compound is, for example, described in the Merck index, 10th edition, page 1025, Merck & Co, Inc, 1983. Other names used to describe this compound are, inter alia, xylan hydrogen sulfate; xylan polysulfate; CB 8061; Fibrase; Hemoclar.

As used herein, the term “biomarkers” is intended as art recognized and refers to molecules or compounds, e.g., protein or gene, whose presence or levels indicates the presence of a disease or the heightened likelihood of developing a disease, e.g., cancer. For example, patients that have elevated levels of prostate specific antigen are likely to have or to develop prostate cancer. Patients that have a mutation in BRCA1 or BRCA2 genes are likely to have or to develop breast and ovarian cancers.

As used herein, the term “ex vivo” refers to tests performed using living cells in tissue culture.

Telomerase Inhibitors

As noted above, the immortalization of cells often involves, inter alia, the activation of telomerase. More specifically, the connection between telomerase activity and the ability of many tumor cell lines, including skin, connective tissue, adipose, breast, lung, stomach, pancreas, ovary, cervix, uterus, kidney, bladder, colon, prostate, central nervous system, retina and blood tumor cell lines, to remain immortal has been demonstrated by analysis of telomerase activity (Kim et al., 1994, Science, 266:2011-2014). This information, supplemented by the prior art indicating that the shortening of telomere length can provide the signal for apoptosis, or replicative senescence (WO 93/23572), indicates that inhibition of telomerase activity for a sufficiently long time can be an effective anti-cancer therapy.

In a related embodiment, the invention is a method for inhibiting the ability of a cell to proliferate or replicate. In this method, one or more compounds described in the present invention and that is capable of inhibiting telomerase enzyme activity, are provided during cell replication. As explained above, telomeres play a critical role in allowing the end of the linear chromosomal DNA to be replicated completely without the loss of terminal bases at the 5′-end of each strand. Immortal cells and rapidly proliferating cells use telomerase to add telomeric DNA repeats to chromosomal ends. Inhibition of telomerase will result in the proliferating cells not being able to add telomeres and they will eventually stop dividing. As will be evident to those of ordinary skill in the art, this method for inhibiting the ability of a cell to proliferate is useful for the treatment of a condition associated with an increased rate of proliferation of a cell, such as in cancer (telomerase-activity in malignant cells), and hematopoiesis (telomerase activity in hematopoietic stem cells), for example.

Thus, in one aspect, the present invention teaches using suramin, PPS, and hTR-antisense to inhibit telomerase, and thereby preventing or treating many types of malignancies. In particular, the compounds of the present invention can provide a highly general method of treating malignancies, as demonstrated by the high percentage of human tumor cell lines and tumors that express telomerase. More importantly, the compounds described in the present invention can be effective in providing treatments that selectively target malignant cells, thus avoiding many of the deleterious side-effects usually associated with cytotoxic chemotherapeutic agents that kill dividing cells indiscriminately.

The compounds of the invention extend to analogues of suramin and PPS that also inhibit telomerase.

In another aspect, the present invention provides compounds, pharmaceutical compositions and methods relating to these compounds, or their pharmaceutically acceptable salts, for inhibiting a telomerase enzyme, comprising contacting the telomerase enzyme with a compound, or its pharmaceutically acceptable salt, where the compounds are suramin or PPS.

In a preferred embodiment, the telomerase to be inhibited is a mammalian telomerase, such as a human telomerase.

Anti-Tumor Activity of the Telomerase Inhibitors

A second and related aspect of the present invention is the discovery that suramin, when maintained in a subject in amounts that are effective in the inhibition of telomerase activity, can shorten the telomere length in a tumor. Thus, this aspect of the present invention teaches methods of inhibiting telomerase activity in a patient, preferably a mammal, suffering from a telomerase-mediated condition or disease, comprising administration to the patient of a therapeutically effective amount of suramin, or another telomerase inhibitor.

The compounds described in the present invention inhibit telomerase in cell extracts, cultured cells and in intact animals. The activities of the compounds of the invention can also be demonstrated using the methods described herein.

One method used to identify compounds of the invention that inhibit telomerase activity involves placing cells, tissues, or preferably a cellular extract or other preparation containing telomerase, in contact with several known concentrations of a test compound in a buffer compatible with telomerase activity. The level of telomerase activity for each concentration of test compound is measured and the IC₅₀ (the concentration of the test compound that produced 50% inhibition of the enzyme activity relative to its original or control value) or IC₉₀ for the compound is determined using standard techniques. Other methods for determining the inhibitory concentration of a compound of the invention against telomerase can be employed as will be apparent to those of skill in the art based on the disclosure herein.

With the above-described methods, IC₅₀ values for suramin and PPS were determined and found to be below 10 μM.

With respect to the treatment of malignant diseases using the compounds described herein, compounds described in the present invention are expected to induce crisis in telomerase-dependent cell lines. Treatment of telomerase-dependent cell lines, e.g., human pharynx FaDu cells, with a compound of the invention is also expected to induce a reduction of telomere length in the treated cells.

Compounds described in the present invention also are expected to induce telomere reduction during cell division in human tumor cell lines, such as FaDu and human prostate PC3. Importantly, however, in normal human cells used as a control, such as BJ cells of fibroblast origin, the observed reduction in telomere length is expected to be no different from cells that are treated only with the vehicle, e.g., physiological saline. Compounds described in the invention also are expected to demonstrate no significant cytotoxic effects in normal cells at the telomerase inhibitory concentrations of their proposed use.

In addition, the specificity of the compounds described in the present invention for telomerase can be determined by comparing their activity (IC₅₀) on telomerase to their activity on other enzymes. Enzymes are, as art recognized, molecules that facilitate a biological reaction. As examples, enzymes similar nucleic acid binding or modifying activity similar to telomerase in vitro include DNA Polymerase I, HeLa RNA Polymerase II, T3 RNA Polymerase, MMLV Reverse Transcriptase, Topoisomerase I, Topoisomerase II, Terminal Transferase and Single-Stranded DNA Binding Protein (SSB). Other enzymes not within this group also are included. Compounds having lower IC₅₀ values for telomerase as compared to the IC₅₀ values toward the other enzymes being screened are said to possess specificity for telomerase. Alternatively, compounds having lower IC₉₀ values for telomerase as compared to the IC₉₀ values toward the other enzymes being screened are said to possess specificity for telomerase.

In vivo testing also can be performed using a mouse xenograft model, e.g. FaDu tumor cells transplanted into nude mice, in which mice treated with a compound of the invention are expected to have tumor masses that, on average, may decrease, remain unchanged, or even increase for a period following the initial dosing, but will shrink in mass with continuing treatment. In contrast, control animals treated with physiological saline are expected to have tumor masses that continue to increase.

From the foregoing those skilled in the art will appreciate that the present invention also provides methods for selecting treatment regimens involving administration of a compound of the invention. For such purposes, it may be helpful to perform a terminal restriction fragment (TRF) analysis in which DNA from tumor cells is analyzed by digestion with restriction enzymes specific for sequences other than the telomeric (T₂AG₃)ₙ sequence. An example of such analysis is described in a previous patent application (U.S. patent application Ser. No. 10/464,018). Following digestion of the DNA, gel electrophoresis is performed to separate the restriction fragments according to size. The separated fragments then are probed with nucleic acid probes specific for telomeric sequences to determine the lengths of the terminal fragments containing the telomere DNA of the cells in the sample. By measuring the length of telomeric DNA, one can estimate how long a telomerase inhibitor should be administered and whether other methods of therapy (e.g., surgery, chemotherapy and/or radiation) should also be employed. In addition, during treatment, one can test cells to determine whether a decrease in telomere length over progressive cell divisions is occurring to demonstrate treatment efficacy.

Telomerase Inhibition Pretreatment Before Chemotherapy

A third aspect of the present invention is the discovery that treatment with suramin prior to the initiation of cancer chemotherapy, in the presence of minimal residual disease and where the suramin is administered in amounts that are effective to inhibit telomerase activity, enhances the efficacy of the cancer chemotherapy. Thus, this aspect of the present invention teaches methods of improving the treatment outcome of a cancer patient, preferably a mammal, comprising administration to the patient of a telomerase-inhibitory amount of suramin, or another telomerase inhibitor. Preferably, the telomerase-inhibiting treatment is given in such a way that the tumor is exposed to a telomerase inhibitor for a duration of at least several cycles of proliferation, and more preferably at least 10 to 20 cycles of proliferation. A preferred method is the administration of the telomerase inhibitor before the administration of a cytotoxic regimen. An alternative preferred method is the administration of the telomerase inhibitor before and during the administration of a cytotoxic regimen. In a preferred embodiment, the telomerase inhibitor is suramin, where the suramin is administered in an amount that is effective in the inhibition of telomerase activity but insufficient to produce antitumor activity.

Continued Telomerase Inhibition After Cytoreductive Therapy

A fourth aspect of the present invention is the discovery that maintenance of suramin in plasma at telomerase-inhibitory concentrations during and after completion of a cytoreductive treatment promoted tumor shrinkage, delayed tumor growth, and extended the survival time in human cancer patients. Thus, this aspect of the present invention provides methods of enhancing the treatment of a cancer patient, preferably a mammal, comprising administering to the patient a therapeutically effective amount of suramin, or another telomerase inhibitor, during and after completion of a cytoreductive treatment. Such cytoreductive treatment can be surgical excision of tumors or non-surgical treatments, e.g., radiation therapy, chemotherapy, photodynamic therapy. Preferably, the telomerase-inhibiting treatment is given in such a way that the tumor is exposed to a telomerase inhibitor at a plasma concentration that is known to produce telomerase inhibition in tumor cells, for a duration that is equivalent to at least several cycles of proliferation, and more preferably at least 10 to 20 cycles of proliferation. A preferred method is the administration of the telomerase inhibitor during and after the administration of a cytotoxic regimen, where the cytotoxic regimen will reduce the tumor load in the patient and thereby provide sufficient lead time for telomerase inhibitors to result in shortening of telomere to below the critical length to induce cell death and senescence. In another embodiment, reduction of the tumor size is accomplished by surgical means. In another embodiment, a telomerase inhibitor is administered after a cytotoxic regimen is terminated, e.g., due to dose-limiting toxicity in the cancer patient, as a means to combat cancer. Preferably, a telomerase inhibitor is administered after a cytotoxic regimen is terminated and the administration of a telomerase inhibitor is continued for at least several weeks, months, years, or more preferably, indefinite period.

Applicants discovered that suramin and PPS sensitize tumor cells to cytotoxic treatments, such as cancer chemotherapy and radiation (U.S. patent application Ser. No. 09/587,559; Example 10). Hence, either of these compounds, or the combination of these two compounds, can be used to sensitize the tumor cell to a cytotoxic anti-cancer treatment, while at the same time providing the additional benefit of inhibiting the telomerase activity, and hence promoting the shortening of the telomeres. After completion of the cytotoxic cancer treatment regimen, treatment with suramin, and PPS, or another inhibitor of telomerase activity, is continued. This double treatment benefit of suramin and PPS represents an unexpected advantage of a treatment using these compounds.

Chemoprevention By Telomerase Inhibition

In a fifth aspect, the invention teaches a method of treating a cancer in a patient by identifying a patient about to have a cancer, or harboring a cancer that is too small to be detected by conventional means, and administering a telomerase inhibitory agent to the patient such that treatment of the cancer or prevention of cancer development is achieved. As examples, some of the conventional methods to detect tumors are physical methods (e.g., palpation), pathological methods (e.g., blood in urine or stool), or imaging methods (e.g., X-ray, CAT scan, PET scan, ultrasound sonogram). Identification of patients that are likely to have a cancer or harboring an undetectable cancer can also be achieved by monitoring biomarkers or genetic defects. For example, a patient may have a blood level of the prostate specific antigen (PSA) that is above the normal limit of 4 ng/ml, but may not have a tumor palpable by digital rectal exam, or visible by ultrasound imaging. The elevated PSA level would indicate a high likelihood of the formation or the presence of a cancer of the prostate, while the absence of physical detection indicates that the tumor is extremely small, or in a precursor state. As another example, a female patient, not currently having a detectable tumor, could have a mutation in the BRCA1 or BRCA2 gene, showing a strong predisposition for the development of a breast or ovarian cancer. Other art recognized methods for assessing the likelihood of tumor occurrence and the tumor burden are also included. Treatment of such patients with a telomerase inhibitory agent would be effective, as the small initial size of the tumor would allow many tumor proliferation cycles before the tumor burden would threaten the health and well being of the patient.

In a sixth aspect, the invention is a method of treating a patient in remission, after a successful treatment of his or her cancer, but where the patient remains at a substantial risk of developing a new or recurrent cancer. For example, a patient treated for disseminated diffuse large B-cell lymphoma with a regimen of intravenous cyclophosphamide, 750 mg/m², doxorubicin, 50 mg/m², vincristine, 1.4 mg/M², and oral prednisone, 100 mg/m², and found to respond slowly to this treatment, eventually achieving remission, would have a statistically high chance of contracting a recurrence of the cancer (Armitage, et al., J Clin. Oncol., 4:160-164, 1986). This patient would benefit from a treatment with a telomerase inhibitor. The telomerase inhibitor would effectively combat the patient's cancer if it reappears. This is especially so for an agent of minimal or no toxicity to the patient, as it provides for a favorable risk-to-benefit ratio. Suramin, PPS, or AZT, inter alia, at the low amounts needed to inhibit telomerase activity, are minimally or not toxic to the patient, and provide such favorable risk-to-benefit ratio.

Telomerase Inhibition With hTR Antisense

A seventh aspect of the present invention is based on the Applicants' finding that transfection of cells with the hTR-antisense effectively inhibits the telomerase enzyme activity in vitro. The nucleotide sequence defining the hTR-antisense is obvious from Example 4. Thus, the present invention teaches methods of inhibiting telomerase by transfecting cells with the hTR-antisense.

Low-Dose AZT

In an eighth aspect, the invention teaches the use of nontoxic doses of AZT yielding plasma concentrations below the micromolar range, e.g., in the nanomolar range, to combat cancer. In one embodiment, AZT is given to a cancer patient, without other accompanying treatment. In another embodiment, AZT is given to a cancer patient after a surgical cytoreductive treatment. In another embodiment, AZT is given prior to, concurrently with, or after a nonsurgical cytoreductive treatment.

Local Administration of Telomerase Inhibitors to a Target Site or Organ

In a ninth aspect, the telomerase inhibitor can be administered locally or regionally, near the site of a known tumor, or in an organ where the current or future occurrence of a tumor is suspected. The locally administered telomerase inhibitor will provide effective inhibitory concentrations to tumor cells or tissues located in proximity to the administration site. Local administration, e.g., injection (e.g., intraluminally) or implantation, can be in the form of a depot, such as a slow release device. Implantation may require a surgical procedure, depending on the site of the treatment. For example, a patient successfully treated for a superficial bladder cancer of high grade would have no known remaining tumor, but would be at an elevated statistical risk of tumor recurrence. In such a patient, a device that slowly releases a telomerase inhibitor over a period of weeks or months or years could be placed directly into the urinary bladder by transurethral insertion. The released telomerase inhibitor would provide effective inhibitory concentrations to the mucosal or muscle cell layers of the urinary bladder, and thus inhibit tumor formation and eventually any recurring tumor. As another example, in a patient with elevated prostate specific antigen (PSA) concentrations and hence suspected prostate cancer, local administration or implantation of a slow-release preparation in or near the prostate to deliver one or more telomerase inhibitors can be used to reduce the chance of the development of symptomatic tumors.

Local administration of the telomerase inhibitor is to provide telomerase-inhibitory concentrations in the tissues that are targets of the treatment, but does not need to result in telomerase-inhibitory concentrations in plasma or other organs that are not the targets of the treatment. This aspect of the present invention is based on the discovery that local administration of suramin to the targeted organ resulted in telomerase-inhibitory concentrations in the targeted tissues (e.g., 5 to 100 μg/g) but very low concentrations of suramin in the plasma (e.g., 0.1 μg/ml or less).

Local administration of the telomerase inhibitor has certain advantages over systemic routes of administration. One advantage is that it avoids the need of frequent drug administrations, while assuring that telomerase inhibition is continuous and uninterrupted. Another advantage is that the local administration diminishes the possibility of toxicity of a telomerase inhibitor to other tissues that are not the intended targets of the treatment. Even though no toxicities were apparent after the systemic administration the low doses of a telomerase inhibitor, e.g. suramin, that are needed to provide telomerase-inhibitory concentrations in human cancer patients (e.g., see Example 7), a reduction of exposure of non-tumor-bearing organs will further reduce the chance that a hypersensitivity reaction, or other rare event, occurs. Considering the application of other telomerase inhibitors, which may exhibit toxicities after systemic administration, this advantage would be of even greater significance.

In another embodiment utilizing PPS (or suramin), the invention can be used to treat bladder interstitial cystitis by use of bladder local administration, e.g., injection or implantation, can be in the form of a depot, such as a slow release device. This method is for enhancing therapeutic outcome of treating patient having bladder interstitial and is implemented by locally administering to the patient an effective amount of one or more of suramin, a pharmaceutically acceptable salt of suramin, pentosan polysulfate (PPS), a pharmaceutically acceptable salt of PPS.

Methods for Inhibiting or Reducing Cell Growth

In one aspect, the invention features methods for inhibiting or reducing cell growth, e.g., aberrant growth, e.g., hyperplastic or hypertrophic cell growth, by contacting the cells with at least one cytoreductive agent and at least one telomerase inhibiting agent. In general, the methods include a step of contacting pathological hyperproliferative cells (e.g., a cancer cell) with an amount of at least one telomerase inhibiting agent which is effective to reduce or inhibit the proliferation of the cell, or induce cell killing.

The present methods can be performed on cells in culture, e.g., in vitro or ex vivo, or can be performed on cells present in a subject, e.g., as part of an in vivo therapeutic protocol. The therapeutic regimen can be carried out on a human or on other animal subjects. The enhanced therapeutic effectiveness of the combination therapy of the present invention represents a promising alternative to conventional highly toxic regimens of anticancer agents.

While the telomerase inhibitory agent can be utilized alone, the agent is preferably combined with a cytotoxic agent for a therapeutic effect that is greater than expected for each of the agents alone. Even further, these agents may be further combined with other anticancer agents, e.g., antimicrotubule agents, topoisomerase I inhibitors, topoisomerase II inhibitors, antimetabolites, mitotic inhibitors, alkylating agents, intercalating agents, agents capable of interfering with a signal transduction pathway (e.g., a protein kinase C inhibitors, e.g., an antihormone, e.g., an antibody against growth factor receptors), agents that promote apoptosis and/or necrosis, biological response modifiers (e.g. interferons, e.g. interleukins, e.g. tumor necrosis factors), surgery, or radiation.

Using the above strategy, the enhanced, and preferably synergistic, action of the cytotoxic agent when used in combination with a telomerase inhibitory agent improves the efficacy of the anticancer agent/s allowing for the administration of lower doses of one or more of these agents (even, e.g., a subtherapeutic dose of an agent, if only tested or used alone rather than in combination); thus, reducing the induction of side effects in a subject, such as a human cancer patient (e.g., any art recognized side effects associated with the administration of an unmodified dose of a chemotherapeutic, e.g., hair loss, neutropenia, intestinal epithelial cell sloughing, etc.).

Methods for Treating Cancer

The methods of the invention can be used in treating malignancies of the various organ systems, such as those affecting lung, breast, lymphoid, gastrointestinal (e.g., colon, rectum), and the genitourinary tract (e.g., prostate, bladder, testes), pharynx, as well as adenocarcinomas which include malignancies such as colon cancer, rectal cancer, renal cell carcinoma, prostate cancer and/or testicular tumors, non-small cell carcinoma of the lung,.cancer of the small intestine, and cancer of the esophagus.

Exemplary solid tumors that can be treated include, e.g., fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, colon carcinoma, rectal cancer, pancreatic cancer, breast cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi's sarcoma.

The methods of the invention also can be used to inhibit or reduce the growth of a cell of hematopoietic origin, e.g., arising from the myeloid, lymphoid, or erythroid lineages, or any precursor cells thereof. For instance, the present invention contemplates the treatment of various myeloid disorders including, but not limited to, acute promyeloid leukemia (APML), acute myelogenous leukemia (AML), and chronic myelogenous leukemia (CML) (reviewed in Vaickus, L. (1991) Crit. Rev. Oncol./Hemotol. 11:267-97). Lymphoid malignancies, which can be treated by the method, include, but are not limited, to acute lymphoblastic leukemia (ALL; which includes B-lineage ALL and T-lineage ALL), chronic lymphocytic leukemia (CLL), prolymphocytic leukemia (PLL), hairy cell leukemia (HLL), and Waldenstrom's macroglobulinemia (WM).

Additional forms of malignant lymphomas contemplated by the treatment method of the present invention include, but are not limited to, non-Hodgkin's lymphoma and variants thereof, e.g., peripheral T-cell lymphomas, adult T-cell leukemia/lymphoma (ATL), cutaneous T-cell lymphoma (CTCL), and large granular lymphocytic leukemia (LGF).

Other malignancies, which can be treated by the subject methods, include erythroleukemias, lymphomas, Hodgkin's disease, and malignancies of uncertain origin, e.g., which are not easily categorized and may, e.g., exhibit multiple cell types, such as certain embryonic carcinomas or teratomas.

For example, the subject can be a patient with non-small cell lung cancer, and is treated with a combination of paclitaxel, carboplatin, and long-term suramin, where the suramin treatment is continued well after the paclitaxel/carboplatin combination needed to be discontinued for reasons of drug-related toxicity or because the patient ceased to respond. Alternatively, a patient with non-small cell lung cancer can be treated with a combination of gemcitabine, cisplatin, and long-term suramin.

In another example, the subject can be a patient with hormone refractory prostate cancer, who is treated with a combination of estramustine phosphate, taxotere, and long-term suramin, or with a combination of doxorubicin, ketoconazole, and long-term suramin.

In still another example, the subject can be a patient with metastatic breast cancer, who is treated with a combination of cyclophosphamide, doxorubicin, 5-fluorouracil, and long-term suramin, or a combination of doxorubicin, taxotere, and long-term suramin. In a related example, the subject is a patient with advanced breast cancer that overexpresses the HER2/neu oncogene, who is treated with Herceptin and long-term suramin, with or without paclitaxel or cisplatin.

In still another example, the subject can be a patient with advanced or metastatic colorectal cancer, who is treated with a combination of irinotecan and long-term suramin. In a related example, the subject is a patient with advanced colon cancer, who is treated with a combination of 5-fluorouracil, leucovorin, and long-term suramin.

Methods of Administration

In a preferred embodiment of the invention, the telomerase inhibitory agent is administered systemically. For example, the selected agent can be administered parenterally (e.g., subcutaneously, intravenously, intramuscularly, intraperitoneally, intradermally, intrathecally, intraluminally etc.), orally, nasally, intrapulmonary by inhalation, rectally, and/or transdermally.

In another embodiment, the telomerase inhibitory agent is administered locally or regionally. For example, the selected agent can be administered intravesically (i.e., into the urinary bladder), intraprostatically, intratumorally, or topically.

In another embodiment, the method further includes repeated dosages of the same, or a different agent, and such particulars are further discussed below.

Dosage Regimens

A physician or veterinarian having ordinary skill in the art can readily determine and prescribe the effective amount of the agent or agents (e.g., in the form of a pharmaceutical composition) required. For example, the physician or veterinarian typically may start doses of the agents of the invention at levels lower than that required in order to commence the desired therapeutic effect and gradually increase the dosage until the desired effect is achieved.

In general, a suitable dose of an agent of the invention will be that amount of the agent which is the lowest dose effective to produce a therapeutic effect; i.e., treat a condition in a subject, e.g., cancer. Such an effective dose will generally depend upon the factors described above. Generally, intravenous and subcutaneous doses of the agents of this invention for a patient, will range from about 0.0001 to about 100 mg per kilogram of body weight, more preferably from about 0.01 to about 10 mg per kg, and still more preferably from about 0.10 to about 4 mg per kg. If desired, the effective daily dose of the active agent may be administered as two, three, four, five, six, or more sub-doses administered separately at appropriate intervals throughout the day, optionally, in unit dosage forms.

In a preferred embodiment, the telomerase inhibitor is suramin and the suramin is present in a concentration that is sufficient to inhibit telomerase activity, but is not sufficient to produce one or more of: (i) significant inhibition of cell proliferation; (ii) significant cell death in human and/or animal tumor cells, (iii) a measurable antitumor effect in a subject, e.g., a human subject, and/or (iv) cell cycle arrest. The determination of effect on cultured cells can be determined with the system described in Example 14.

In a preferred embodiment, the telomerase inhibitor is suramin and it is administered at levels such that the plasma concentration of suramin that is present does not result in one or more of: (i) significant cell cycle arrest, (ii) significant cell death, or (iii) significant inhibition of cell growth, e.g., the concentration in plasma is of a level that, if the same concentration of suramin is provided in cultured cells, at least 50%, more preferably at least 80%, and most preferably at least 99% of the treated cultured cells continue to be involved in one or more of: cycling cells continue to progress through the cell cycle, cells remain viable, or cells remain capable of proliferating, following treatment with suramin.

Preferably, suramin is administered in an amount that results in a plasma concentration ranging from about 0.001 to 100 μg/ml, preferably about 0.1 to 70 μg/ml, even more preferably, about 0.5 to 30 μg/ml. The pharmacokinetics of suramin is characterized by a triphasic concentration decline, with half-lives of 5.5 hours, 4.1 days and 78 days. The total body clearance is 0.0095 liter/hour/m² (Jodrell et al., J Clin Oncol 12:166-175, 1994). Based on pharmacokinetic principles, a person skilled in the art can calculate that an initial dose of approximately 240 mg/m² should be administered to the average patient to achieve plasma concentrations declining from about 90 μg/ml (63 μM) to about 14 μg/ml (10 μM) over 168 hours. The 168-hour, or 1 week, duration is chosen as an example, as this time interval is frequently used for repeat visits to a treating physician. Similar calculations can be performed to identify the initial suramin dose to deliver the preferred suramin plasma concentrations over other desired treatment durations. Maintenance doses to adjust the plasma concentrations for later treatment cycles can be similarly calculated.

In a preferred embodiment, the total suramin exposure in the plasma preferably is less than 7,840 μM-day over 112 days, less than 7,100 μM-day over 112 days, less than 5,880 μM-day over 84 days, less than 5,300 μM-day over 84 days, less than 2,000 μM-day over 20 days, preferably less than 800 μM-day over 96 hours, preferably less than 600 μM-day over 96 hours, preferably less than 500 μM-day over 96 hours, preferably less than 400 μM-day over 96 hours, preferably less than 300 μM-day over 96 hours, preferably less than 252 μM-day over 96 hours, preferably less than 200 μM-day over 96 hours, preferably less than 150 μM-day over 96 hours, preferably less than 100 μM-day over 96 hours, and most preferably less than 52 μM-day over 96 hours. The total suramin exposure, as expressed in μM-day, is a product of the drug plasma concentration in μM-day (e.g., the average micromolarity over 24 hours) and the treatment duration in days. For example, treatment of a subject with 13 μM of suramin for four days would result in a total drug exposure of 52 μM-day over 96 hours.

Preferably, suramin is administered in an amount that results in a plasma concentration of less than 100 μg/ml, preferably less than 90 μg/ml, preferably less than 80 μg/ml, preferably less than 60 μg/ml, preferably less than 40 μg/ml, more preferably less than 15 μg/ml, and most preferably less than 10 μg/ml.

In a preferred embodiment, the telomerase inhibitor is suramin and the time period over which the suramin is administered or over which the suramin is maintained at the plasma concentration sufficient to inhibit telomerase activity is more than one month, or preferably more then one year, or even more preferably indefinitely.

In a preferred embodiment, the telomerase inhibitor is suramin and the time period over which the suramin is administered or over which the suramin is maintained at the plasma concentration sufficient to inhibit telomerase activity is longer than 60 days, preferably longer than 100 days, preferably longer than 150 days, preferably longer than one year, more preferably longer than two years, and most preferably for indefinite time period, beyond the time duration where a cytoreductive treatment is applied.

In a preferred embodiment, the telomerase inhibitor is suramin and suramin is administered locally to the target organ, and the time period over which the suramin concentration in the tissues intended for suramin treatment is sufficient to inhibit telomerase activity is more than one month, or preferably more than one year, or more preferably indefinite. For example, when using suramin as a cancer preventative, treatment may require years of use stretching to the rest of the patient's life.

In a preferred embodiment, the telomerase inhibitor is suramin and suramin is administered locally to the target organ, and the time period over which the suramin concentration in the tissues intended for suramin treatment is sufficient to inhibit telomerase activity is longer than 60 days, preferably longer than 100 days, preferably longer than 150 days, preferably longer than one year, more preferably longer than two years, and most preferably for indefinite time period, beyond the time duration where a cytoreductive treatment is applied.

In a preferred embodiment, the telomerase inhibitor is suramin and the time period over which the suramin is administered or over which the suramin is maintained at the plasma concentration sufficient to inhibit the telomerase activity or to enhance the efficacy of the cytoreductive treatment begins more than 30 days, preferably more than 60 days, preferably more than 100 days, preferably more than 150 days, preferably more than one year, and most preferably more than two years before the first day on which the cytoreductive treatment is administered.

Methods described herein use suramin to enhance the antitumor effect of a cytoreductive treatment, where the suramin dose is selected to deliver a plasma concentration of below 100 μg/ml, preferably below 80 μg/ml, preferably below 60 μg/ml, more preferably below 40 μg/ml, and most preferably below 15 μg/ml in a mammal treated with a cytoreductive treatment. The suramin dose is administered before, simultaneously with, or after the administration of at least one anticancer agent or other cytoreductive treatment. Animal trials presented herein show that treatment of mice with two weekly intravenous bolus suramin doses of 10 mg/kg for 6 weeks enhances the antitumor effect of the subsequently administered anticancer drugs (e.g., paclitaxel), but does not result in additional body weight loss. This dose is calculated to result in a plasma suramin concentration of about 10 μM (˜14 μg/ml) at 72 hours after dose administration. The methods of the art use high dose suramin, either alone or in combination with a cytotoxic agent, where for a human subject, maintenance of plasma suramin concentrations of between 150 to 300 μg/ml is needed to produce a measurable antitumor effect (Eisenberger et al., (1995) J Clin Oncol 13:2174-2186; Klohs, U.S. Pat. Nos. 5,597,830 and 5,767,110). A typical suramin dosing schedule aimed at maintaining suramin plasma concentrations between 150 and 300 μg/ml consists of an initial administration of 2100 mg/m² over the first week with the subsequent doses repeated every 28 days for 6 months or longer; the subsequent doses are adjusted using the Bayesian pharmacokinetic method (Dawson et al., Clin Cancer Res 4:37-44, 1998; Falcone et al., Cancer 86:470-476, 1999). At these doses and chronic treatments, suramin causes the following toxicity in a human patient: adrenal insufficiency, coagulopathy, peripheral neuropathy, and proximal muscle weakness (Dorr and Von Hoff, Cancer Chemotherapy Handbook, 1994, pp 859-866). The incidence and severity of these toxicities are positively related to cumulated dose and are minimized in the methods described herein.

In a preferred embodiment, the telomerase inhibitor is PPS. Preferably, the PPS is present in a concentration that is sufficient to inhibit telomerase activity and induce shortening of telomeres in tumor cells, but is not sufficient to produce one or more of: (i) significant anti-coagulation activity; (ii) significant cell death in human and/or animal tumor cells, (iii) a measurable antitumor effect in a subject, e.g., a human subject, and/or (iv) cell cycle arrest.

While it is possible for an agent of the present invention to be administered alone or in combination with another agent, it is preferable to administer the agent(s) as a pharmaceutical composition.

In a preferred embodiment, the telomerase inhibitory agent is suramin.

Compositions and Formulations

In another aspect, the invention features a pharmaceutical composition, which includes at least one telomerase inhibitory agent and a pharmaceutically acceptable carrier. Preferably, the agent(s) are present in an amount effective to inhibit the telomerase activity in the tumor of the patient, and to enhancing the killing, of a hyperproliferative cell.

In a preferred embodiment, the pharmaceutical composition or compositions are packaged with instructions for use as described herein.

The invention also encompasses timed-release formulations, for example, a slow release formulation of a telomerase inhibitory agent, and a pharmaceutically acceptable carrier.

In another embodiment, the pharmaceutical composition is suitable for intravenous injection. The composition may also be suitable for local, regional, or systemic administration.

In another embodiment, the pharmaceutical composition may comprise one or more pharmaceutically acceptable carriers. In yet another embodiment, the invention pertains to nanoparticles, which comprise a cross-linked gelatin and a therapeutic agent, e.g., a telomerase inhibitory agent, such as, for example, suramin or PPS. In a further embodiment, the invention pertains to compositions containing the nanoparticles and a pharmaceutically acceptable carrier. The carrier, for example, can be suitable for systemic, regional, or local administration. In one embodiment, the nanoparticles are about 500 to about 1 μm, or about 600 nm to about 800 nm in diameter.

The invention also pertains to microparticles comprising a therapeutic agent, e.g. a telomerase inhibitory agent, such as suramin or PPS. In one embodiment, the size of the microparticle ranges from about 10 nm to about 300 μ and in another embodiment from about 10 nm to about 300 nm. In another embodiment, the invention pertains to a composition, which comprises the microparticles and a pharmaceutically acceptable carrier. The pharmaceutically acceptable carrier may be, for example, suitable for administration to a patient locally, regionally, or systemically. The invention also pertains to a method for treating a patient, comprising administering to the patient microparticles of the invention and a pharmaceutically acceptable carrier.

In another embodiment, the invention features a microparticle suitable for administration to a patient locally, regionally, or systemically, comprising paclitaxel, wherein said microparticle has a diameter of about 5μm. In another further embodiment, the invention features microparticles suitable for administration to a patient locally, regionally, or systemically, comprising suramin or PPS, wherein said microparticle has a diameter of about 5 μm.

The invention also pertains to a kit, i.e., an article of manufacture, for the treatment of a cancer. The kit contains a telomerase inhibitory agent in a pharmaceutically acceptable carrier, a container, and directions for using said telomerase inhibitory agent for inhibiting or reducing the growth of a cell, e.g., aberrant growth associated with, e.g., a cancer or a tumor. For example, a kit of the invention may comprise a telomerase inhibitory agent for previous, subsequent, or concurrent administration. The kit may also provide the telomerase inhibitory agent formulated in dosages and carriers appropriately for local, regional, or systemic administration. Still further, the kit may also provide for the prognosing, diagnosing, and/or staging of a cancer for, e.g., determining the susceptibility or resistance of the cancer.

Pharmaceutical compositions comprising compounds of the invention may contain wetting agents, emulsifiers and lubricants, such as sodium lauryl sulfate and magnesium stearate, as well as coloring agents, release agents, coating agents, sweetening, flavoring, and perfuming agents, and preservatives.

Formulations of the present invention include those suitable for oral, nasal, topical, inhalation, transdermal, buccal, sublingual, rectal, vaginal, and/or parenteral administration. They are given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. The formulations conveniently may be presented in unit dosage form and may be prepared by any methods well known in the art of pharmacy. The amount of active ingredient that can be combined with a carrier material to produce a single dosage form will generally be that amount of the agent that produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or non-aqueous solutions, dispersions, suspensions, or emulsions, or sterile powders which may be reconstituted into sterile injectable solutions or dispersions just prior to use, which may contain buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers, such as, for example, polylactide-polyglycolide. Depending on the ratio of drug to polymer, and the nature of the particular polymer employed, the rate of drug release can be controlled. Examples of other biodegradable polymers include, for example, poly(orthoesters) and poly(anhydrides). Depot injectable formulations are also prepared by entrapping the drug in liposomes or microemulsions, which are compatible with body tissue.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient, which is effective to achieve the desired therapeutic response for a particular patient, composition, and mode of administration, without being toxic to the patient.

The selected dosage level will depend upon a variety of factors including, inter alia, the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

### EXEMPLIFICATION OF THE INVENTION

The invention now being generally described, it will be more readily understood by reference to the following examples, which are included merely for purposes of illustration of certain aspects and embodiments of the present invention, and are not intended to limit the invention.

Throughout the examples, unless otherwise indicated, the practice of the present invention will employ conventional techniques of chemistry, molecular biology, microbiology, recombinant DNA technology, cell culture, and animal husbandry, which are within the skill of the art and are explained fully in the literature. See, e.g., Sambrook, Fritsch and Maniatis, Molecular Cloning: Cold Spring Harbor Laboratory Press (1989); DNA Cloning, Vols. 1 and 2, (D. N. Glover, Ed. 1985); Harlow and Lane, Antibodies: a Laboratory Manual, (1988) Cold Spring Harbor; Oligonucleotide Synthesis (M. J. Gait, Ed. 1984); Nucleic Acid Hybridization (B. D. Hames and S. J. Higgins, Eds. 1984); the series Methods In Enzymology (Academic Press, Inc.), particularly Vol.154 and Vol. 155 (Wu and Grossman, Eds; and Current Protocols in Molecular Biology, eds. Ausubel et a., John Wiley & Sons (1992)).

Materials and Methods:

General methodologies. The required materials (e.g., drugs, chemicals and reagents, human breast MCF7 cells, pharynx FaDu cells, prostate PC3 cells), the FaDu tumor xenograft in immunodeficient mice, and 3-dimensional tumor histocultures were obtained, prepared and used described in U.S. patent application Ser. No. 09/587,662, and Gan et al., FEBS Letters, 527:10-14, 2002. Measurement of drug effect in cultured cells was as described in U.S. patent application Ser. No. 09/587,662.

Inhibition of telomerase activity in cell lysates and intact cells. The modified Telomeric Repeat Amplification Protocol (TRAP) assay (Gan, et al., Pharm. Res., 18:488-493, 2001) was used to detect telomerase activity in cell lysate. The telomerase activity in intact cells was measured using intracellular TRAP, as follows. Cells (1×10⁵) were washed twice with PBS and centrifuged. The cell pellet was resuspended in 100 μl of serum-free RPMI 1640 containing 5 u/ml of Streptolysin O, 2 μM of TS primer, and 50 μM of dNTP, and incubated at room temperature for 5 min. The enzyme streptolysin O was used to increase the cell membrane permeability to the TS primer. Upon entering a cell, the TS primer was elongated by the intracellular telomerase in situ. The elongated TS primer was then isolated from the cells and used as the template for PCR amplification. Then 200 μl of RPMI 1640 medium containing 10% FBS was added to the cells to stop the permeating process. The mixture was incubated at 30° C. for 30 min to allow the extension of intracellular TS primer by telomerase. The cells were then lyzed and the cell lysates, which contained the already extended TS primer, was directly analyzed by TRAP.

Measurement of telomere length in cultured cells. Two methods were used to measure telomere length. The first method was a solution hybridization based telomere amount and length assay (TALA) (Gan, et al., Pharm. Res., 18:1655-1659, 2001) that measures the mean length of the terminal restriction fragments (TRF). The second method was fluorescence in situ hybridization (FISH) to detect telomere signal and to estimate the approximate length of individual telomere structures at the end of chromosomes. The FISH method is as described in U.S. patent application Ser. No. 09/587,662 and Gan, et al., 2001.

Detection of senescent cells. Senescent cells were identified by 4-galactosidase staining as described (Dimri, et al., 1995).

### EXAMPLE 1

### Suramin and AZT are Effective Telomerase Inhibitors—In Cell Extracts and Cultured Cells

Inhibition of telomerase. Suramin, an agent with mild reverse transcriptase inhibitory activity but not known to inhibit telomerase, was studied in multiple human cancer cell lines, including human pharynx FaDu, human prostate PC3, and human breast MCF7. Its activity was compared with that of AZT.

Treatment Protocol. Treatment with suramin or AZT was initiated after cells were allowed to attach to the growth surface in culture flasks. On the day of experiments, the culture medium was removed and replaced with inhibitor-containing medium. Drug concentrations of 0, 0.1, 1, 5, 10, 50 μM suramin, or 0, 0.1, 1, 10, 100 μM AZT were employed. Telomerase activity in cell lysates and intact cells, and the telomere length, after 7-15 weeks of growth in medium containing suramin or AZT concentrations ranging from 0 to 50 μM, were measured.

Effect of suramin and AZT on telomerase activity. Telomerase activity was inhibited by suramin and AZT in a concentration-dependent manner in human cancer cells. Concentrations resulting in 50% inhibition are shown in Table 1. The concentrations required for 90% inhibition were less than 50 micromolar.

TABLE 1
Inhibition of Telomerase Activity

| | IC₅₀, micromolar (mean ± SD) | | | | |
|---|---|---|---|---|---|
| | MCF7 | | PC3 | | FaDu |
| Inhib- | Cell | Intact | Cell | Intact | Intact |
| itor | Extract | cell | Extract | cell | cell |
| Suramin | 2.8 ± 1.5 | 1.4 ± 1.1 | 1.6 ± 0.7 | 2.3 ± 0.9 | 1.7 ± 1.0 |
| (μM) | | | | | |
| AZT | —^{a} | Not | —^{a} | Not | 2.1 ± 1.3 |
| (μM) | | determined | | determined | |

^{a}This cannot be studied because the activation of AZT to its triphosphate which is the moiety that inhibits telomerase occurs only in intact cells and not in cell extracts.

Effect of suramin and AZT on telomere length. Prolonged treatment (7-15 weeks) with suramin or AZT resulted in 34-55% telomere shortening in FaDu cells, and about 30% shortening in PC3 cells.

Conclusion. Suramin effectively inhibits telomerase at low micromolar concentrations, and behaves similarly to the known telomerase inhibitor AZT.

### EXAMPLE 2

### Suramin is an Effective Telomerase Inhibitor in Tumor-Bearing Animals

Suramin inhibits telomerase and shortens telomeres in vivo. The in vivo effectiveness of suramin as an inhibitor of telomerase activity was evaluated by measuring the telomere length in tumor cells implanted in immunosuppressed mice.

Treatment protocol. FaDu cells (0.5˜1×10⁶ cells in a volume of 100 μl) were implanted subcutaneously in male BALB/c nu/nu mice. These mice received, by intravenous injection into the tail vein, repeated doses of 10 mg/kg suramin. The first dose was administered immediately after tumor implantation, with repeat doses given twice a week thereafter. Tumors were collected after 2 to 6 weeks of suramin treatment. Frozen tissue sections were analyzed for telomere length in individual cells using fluorescent in situ hybridization. About 10 microscope fields at 400-fold magnification were randomly chosen for each section, and the percentage of tumor cells with attenuated or lost telomere signals were counted. The results were compared with control animals receiving physiologic saline injections instead of suramin.

Effect of suramin on telomere length in vivo. Mice treated with suramin or physiologic saline showed an equal tumor establishment rate of 100%, and undistinguishable rates of bodyweight increases. The FISH result showed a gradual shortening of the telomeres of tumor cells over time. In suramin-treated animals, the fraction of cells with a reduced or eliminated telomere signal remained at the control level of approximately 10% for the first two weeks of treatment, increasing to approximately 40% of cells at week 3, 75% at week 4, over 80% at week 5 and 95% at week 6. No change was observed in the tumor cells of saline-treated control animals.

Conclusion. Suramin effectively reduces telomere length in tumors grown in vivo.

### EXAMPLE 3

### PPS is an Effective Telomerase Inhibitor

PPS inhibits telomerase. The inhibition of telomerase activity in FaDu cells after exposure to PPS was studied.

Treatment Protocol. Treatment with PPS was initiated after cells were allowed to attach to the growth surface in culture flasks. On the day of experiments, the culture medium was removed and replaced with inhibitor-containing medium. Drug concentrations of 0, 0.1, 1, 10, 100, 1000 μg/ml of PPS were employed. Telomerase activity was measured by the modified quantitative TRAP assay.

Effect on telomerase activity. Telomerase activity was inhibited by PPS in a concentration-dependent manner in FaDu and SKOV-3 cells. The concentrations resulting in 50% inhibition were 0.56 and 0.60 μg/ml, respectively. The concentrations resulting in 80% inhibition were less than 10 μg/ml for both cells. The concentrations resulting in 90% inhibition were less than 100 μg/ml for both cells.

Conclusion. PPS is an effective inhibitor of telomerase activity in cells. The concentrations at which PPS produces 50% inhibition of telomerase activity are lower than the concentrations required for anticoagulation (above 1 μg/ml).

### EXAMPLE 4

### hTR Antisense Inhibits Human Telomerase

hTR antisense inhibits telomerase. The antisense study consisted of the following steps: (a) construction of a sense and antisense to the RNA portion of the human telomerase (hTR), (b) stable transfection of cells with the hTR antisense (or hTR sense control), and (c) determination of the ability of the hTR antisense to inhibit telomerase activity and induce telomere shortening. The methodologies are detailed in the art, for example, by Mo et al., Cancer Res. 2003.

Antisense and sense constructs. The sense and anti-sense expression plasmids for human RNA portion of telomerase were prepared. The 185 basepair sense and antisense sequences are given below, and were found to agree with the GenBank sequence (accession No. NR_{—}001566). These procedures resulted in 5 clones that contained the hTR fragment. Sequence analysis indicated that one clone was sense, whereas the other 4 clones were antisense. These hTR sense and hTR antisense expression plasmids were then transfected into human pharynx FaDu cancer cells.

Transfection procedures. Transfection of the antisense construct used an IPTG-inducible mammalian expression system. The resulting clones were used for experiments.

Effect of hTR antisense on cell growth. Table 2 summarizes the results which show a slower growth rate for the antisense +IPTG cells (i.e., cells that were transfected by hTR antisense and treated with IPTG to induce the expression of hTR) compared to cells that had either not been transfected with the antisense, not transfected but treated with IPTG, transfected with the sense and treated with IPTG, or transfected with the antisense but without the IPTG induction (i.e., control, +IPTG, +sense +IPTG, and antisense).

Effect of hTR antisense on the cytotoxic effect of Daclitaxel. Two clones of cells that were stably transfected with the hTR antisense were studied. The cytotoxic effect of paclitaxel was quantified using the SRB method, which measures the total cellular proteins. The cells transfected with hTR antisense were treated with IPTG for 44 (clone#1) to 57 (clone #2) days, and then with paclitaxel for 96 hours. The results, summarized in Table 2, show that the hTR antisense inhibits telomerase activity, and enhances the paclitaxel cytotoxicity in both clones by about 2-fold, as indicated by the reduced IC₅₀ of paclitaxel in the antisense-transfected cells compared to the other control cells.

TABLE 2
Effect Of hTR Antisense On Cell Growth, Paclitaxel Cytotoxicity,
Telomere Length, And Telomerase Activity

| | | | +sense | | +antisense |
|---|---|---|---|---|---|
| Effects | Control | +IPTG | +IPTG | +antisense | +IPTG |
| Doubling time, hr | 22 | 23 | 23 | 23 | 27 |
| IC₅₀ of paclitaxel, | 2.04 | 2.42 | 2.56 | 2.46 | 1.30 |
| nM, clone #1 | | | | | |
| IC₅₀ of paclitaxel, | 2.05 | 2.53 | 2.21 | 2.87 | 1.33 |
| nM, clone #2 | | | | | |
| Terminal restriction | 2.73 | 2.91 | 2.72 | 2.89 | 1.75 |
| fragment, kb | | | | | |
| Telomerase activity, | 100 | 98.9 | 98.2 | 96.5 | 27% |
| % of control | | | | | |

185 bp antisense hTR sequence:

```
5′:
    1 cagctgacattttttgtttgctctagaatgaacggtggaaggcggcaggccgaggctttt
  61 ccgcccgctgaaagtcagcgagaaaaacagcgcgcggggagcaaaagcacggcgcctacg
121 cccttctcagttagggttagacaaaaaatggccaccacccctcccaggcccaccctccgc
181 aaccc
3′
```

185 bp sense hTR sequence:

```
5′:
    1 gggttgcgga gggtgggcct gggaggggtg gtggccattt tttgtctaac cctaactgag
  61 aagggcgtag gcgccgtgct tttgctcccc gcgcgctgtt tttctcgctg actttcagcg
121 ggcggaaaag cctcggcctg ccgccttcca ccgttcattc tagagcaaac aaaaaatgtc
181 agctg
3′
```

Effect of hTR antisense on telomere length and telomerase activity. Telomere length (referred to as terminal restriction fragment) was measured using the TALA method. Telomerase activity was measured using the improved TRAP method. The results, summarized in Table 2, show that the hTR antisense reduced the telomere length and telomerase activity.

Conclusion. Taken together, these results indicate that the treatment of a human cancer cell with a hTR antisense results in an inhibition of telomerase activity, a loss of telomeres, inhibition of cell growth, and enhancement of paclitaxel cytotoxicity.

### EXAMPLE 5

### Administration of Telomerase-Inhibiting Amounts of Suramin Causes Tumor Size Reduction in Animals

Tumor size reduction after low-dose suramin in some animals. The effect of low-dose suramin administration on tumor size was determined in immunosuppressed mice implanted with FaDu tumors.

Treatment Protocol. Immunosuppressed mice were injected subcutaneously in the thigh area with 5×10⁵ FaDu cells. Suramin (10 mg/kg) was administered by intraperitoneal injection, twice-weekly for 6 weeks. Suramin administration was initiated on the day of tumor cell inoculation. The control animals were treated identically, except the suramin solution for injection was replaced by a physiologic saline solution. Tumor sizes were observed twice-weekly.

Effect of low-dose suramin treatment of tumor growth. Six animals received suramin. In five animals, tumor size increased with time. The tumor in one of the remaining animals initially grew, reaching a size of about 4 mm after 2 weeks, but then declined, and completely disappeared by 6 weeks.

Conclusion. Long-term treatment with suramin, a telomerase inhibiting agent, can cause complete disappearance of the tumor in some hosts.

### EXAMPLE 6

### Pretreatment With Low-Dose Suramin Enhances the Antitumor Activity of Chemotherapy in Tumor-Bearing Animals

This example describes the enhancement of the antitumor effect of a chemotherapy agent after prolonged pre-treatment with a telomerase inhibitor. Telomerase inhibition treatment was initiated when the tumor burden is low and not yet palpable, comparable to situation of minimal or undetectable tumors.

Treatment protocol. Female athymic nude mice of 6-8 weeks old were injected subcutaneously in the thigh area with 5×10⁵ viable FaDu cells. Suramin (10 mg/kg) was administered by intraperitoneal injection, twice-weekly for 6 weeks. Suramin administration was started on the day of tumor cell inoculation, when the tumor was of a very small size, representing minimal disease. Control animals were treated identically, except the suramin solution for injection was replaced by a physiologic saline solution. After the 6 week pre-treatment period, suramin treatment was discontinued, and all animals received paclitaxel, 10 mg/kg, twice a week, for three weeks. Tumor sizes were observed twice-weekly, and recorded during the three weeks of paclitaxel treatment.

Effect of low-dose suramin pretreatment. As shown in Table 3, control animals, which received 6 weeks of saline pretreatment, showed an increased tumor size after 3 weeks, whereas the animals receiving suramin pretreatment showed a decline in tumor size.

TABLE 3
Effect of suramin pre-treatment on tumor sizes in mice treated with paclitaxel.

| | Tumor size at different times after initiation of paclitaxel treatment | | | | | | |
|---|---|---|---|---|---|---|---|
| | (% of initial tumor size, Mean ± SD) | | | | | | |
| Treatment | 0 | 0.5 weeks | 1 week | 1.5 weeks | 2 weeks | 2.5 weeks | 3 weeks |
| Saline → paclitaxel | 100 ± 0 | 148 ± 23 | 169 ± 46 | 196 ± 47 | 188 ± 46 | 116 ± 113 | 143 ± 126 |
| Suramin → paclitaxel | 100 ± 0 | 105 ± 56 | 75 ± 106 | 84 ± 119 | 69 ± 98 | 66 ± 93 | 38 ± 53 |

### EXAMPLE 7

### Maintenance of Low, Telomerase-Inhibitory Concentrations of Suramin Promotes Tumor Shrinkage, Delays Tumor Growth and Prolongs Survival of Human Cancer Patients

Human patients with pathologically confirmed, advanced, metastatic, stage IIIB/IV nonsmall cell lung cancer were treated with paclitaxel, carboplatin, and suramin. Treatment was administered about every 3 weeks. The loading dose of suramin was approximately 240 mg/m² and the subsequent doses were calculated based on a mathematical equation Applicants have developed (PCT Application No. PCT/US02/30210). These suramin doses resulted in plasma concentration between about 2 to about 90 micromolar over at least 21 days. As shown in Example 1, these concentrations are sufficient to inhibit telomerase. A total of 54 patients were treated. The first 6 patients received suramin as a single dose, and the remaining patients received suramin in two split doses given 24 hours apart. No toxicity attributed to the use of suramin was observed. Forty-nine patients were evaluable. The overall response rate was 40.8% (consisting of 6% complete response which corresponds to no measurable disease and 34.8% partial response which corresponds to at least 50% tumor shrinkage), the time to disease progression (TTP) was longer than 6 months, and the median survival time (MST) was longer than 12 months (Villalona-Calero, et al., Clin. Cancer Res., 9:3303-3311, 2003; Villalona-Calero, et al., IASLC Meeting, Vancouver, August, 2003).

A comparison of these clinical results to previous clinical trials for paclitaxel/carboplatin in similar patients with advanced, metastatic stage IIIB/IV nonsmall cell lung cancer, indicates that the addition of suramin significantly enhanced the antitumor activity of paclitaxel/carboplatin. For example, a recently completed trial in 290 patients indicates an overall response rate of about 17% (with only <1% patients achieving complete response), TTP of 3.1 months, and MST of 8.1 months (Schiller et al., New Eng J Med, 346:92-98, 2002).

Conclusion. Maintenance of suramin at telomerase-inhibitory plasma concentrations for a long duration, e.g., 12 to 30 weeks, enhances the antitumor activity of paclitaxel and carboplatin and improves the response rate, delays tumor progression and prolongs survival of human cancer patients.

### EXAMPLE 8

### Suramin Has a Long Plasma Halfuramin Life in Dogs

Suramin pharmacokinetics in dogs. The plasma pharmacokinetics of suramin was studied in four beagle dogs.

Treatment protocol. Four beagle dogs, weighing 11.4±0.4 kg, were used. The animals were cannulated in the cephalic veins of both front legs. Suramin (6.75 mg/kg) was infused intravenously over 30 minutes into one vein, while blood samples were obtained from the other vein. Suramin was administered as an aqueous solution of sodium suramin. Blood samples were taken at 5, 30 minutes, 1, 2, 4, 6, 9, 12, 24, 48, 72 hours, and on day 7, 14, and 21, placed in heparinized tubes, and plasma prepared by centrifugation. Plasma concentrations of suramin were determined using high performance liquid chromatography, as previously described (Kassack, et al., J Chromatogr. B Biomed. Appl., 686:275-284,1996). Non-compartmental pharmacokinetic analysis was performed by standard means (Gibaldi, et al., Pharmacokinetics., 1982).

Results. In dogs, suramin is slowly eliminated, with a total clearance of 2.1±0.2 ml/hr/kg, and a terminal half-life of 13.0±3.8 day.

Conclusion. Suramin is slowly eliminated in dogs with an unusually long elimination half-life of 13 days.

### EXAMPLE 9

### PPS Enhances Antitumor Activity of Chemotherapy

This Example teaches that a second telomerase inhibitor, pentosan polysulfate (PPS), enhances the antitumor activity of a cytotoxic agent in cultured tumor cells and primary cultures of patient tumors. The chemosensitization effect of PPS occurs at the telomerase-inhibitory concentrations of 10 and 100 μg/ml, which are >10-fold and >100-fold lower than the PPS concentrations that produce antitumor activity (Wellstein, et al., J. Natl. Cancer Inst., 83:716-720, 1991; Zugmaier, et al., Ann. NY Acad. Sciences, 886:243-248,1999).

A study was performed using two renal cell carcinoma cells (RCC45 and RCC54). 5-Fluorouracil was used as the chemotherapeutic agent. Cells were treated with 5-fluorouracil for 96 hours, with and without PPS. The drug effect was measured as inhibition of the incorporation of a DNA precursor (bromodeoxyuridine or BrdU) using ELISA. The results show that PPS had no cytotoxicity at 10 and 100 μg/ml; the IC₅₀ of PPS, as a single agent, was ˜1,400 μg/ml. Table 4 shows that the IC₅₀ values (i.e., drug concentrations required to produce 50% inhibition) of 5-fluorouracil was reduced by the addition of PPS. Results in Table 4 further show that addition of a second telomerase inhibitor, suramin at a telomerase-inhibitory concentration (i.e., 30 μM), further reduced the IC₅₀ of 5-fluorouracil, indicating that the chemosensitization effect of telomerase inhibitors are additive.

TABLE 4
Nontoxic Concentrations of PPS Enhances the Activity of 5-fluorouracil

| | IC₅₀ of 5-fluorouracil, μM | | | |
|---|---|---|---|---|
| | | | | With 10 μg/ml |
| | | With 10 μg/ml | With 100 μg/ml | PPS plus 30 μM |
| Cell Line | No PPS | PPS | PPS | suramin |
| RCC45 | 7.58 | 5.15 | Not available | Not available |
| RCC54 | 7.27 | 4.65 | 3.58 | 2.34 |

### EXAMPLE 10

### The Telomerase Inhibitor AZT Enhances the In Vivo Antitumor Effect of Chemotherapy Agents at a Surprisingly Low Concentration

This example describes the enhancement of the antitumor effect of a chemotherapeutic (i.e., paclitaxel), by the telomerase inhibitor AZT, in immunodeficient mice bearing human head and neck cancer FaDu xenografts.

The activity of paclitaxel, with or without AZT, was evaluated in immunodeficient mice (male Balb/c nu/nu mice, 6-8 weeks old) bearing the human pharynx FaDu xenografts. Xenografts were formed by subcutaneous injection of 106 viable tumor cells in 0.1 ml physiologic saline in the right and left flank areas, and were allowed to grow for about 14 days to reach a size of >15 mm³ before drug treatment was started. The four treatment groups were: saline control, AZT, paclitaxel, paclitaxel +AZT. The saline control group received injections of 200 μl/day of physiological saline for five consecutive days. The paclitaxel group received injections of 10 mg/kg/day paclitaxel dissolved in Cremophor and ethanol (i.e., Taxol) in a volume of 200 μl for five consecutive days. The AZT group received a seven-day infusion of AZT at a rate of 200 ng/hour by a subcutaneously implanted Alzet minipump. The paclitaxel +AZT group received the combined treatment of the paclitaxel group and the AZT group, where the AZT infusion was started one day prior to the start of the paclitaxel injections. Animal weights and tumor sizes were measured on days 1, 3, 6, 8, and 10 after initiation of the paclitaxel treatment.

The antitumor effect of the drug treatments was measured in three ways. The first was the reduction in tumor size. Tumor sizes were determined by first preparing a mold of the extruding tumor using Jeltrate, a rapidly setting molding material, and then preparing and weighing the countermold. Second, the apoptotic effect was measured. The animals were euthanized on day 10, and the tumors were harvested and fixed in formalin. Histologic sections of 5-micron thickness were prepared and stained with hematoxylin and eosin. The tumor sections were evaluated morphologically for tumor cell density, and density of apoptotic cells. Because apoptotic cells disappear over time, the density of non-apoptotic cells is a secondary indicator of apoptosis. Cell densities were determined by counting the number of cells in four randomly selected microscopic fields at 400× magnification, using image analysis procedures (Song, et al., Proc. Natl. Acad. Sci. USA, 97:8658-8663, 2000). Third, the ability of drug treatment to prolong the survival time was measured. For this study, the animals were monitored for 100 days, or until moribundity, defined by a tumor length exceeding 1.0 cm, was reached. The concentration of AZT in plasma was determined in a parallel experiment, using three mice, subcutaneously implanted with an Alzet 1002 osmotic minipump. Drug infusion was allowed to take place for four days before the blood of the animals was harvested. This long duration of infusion guaranteed that constant steady-state plasma concentrations had been achieved. AZT concentrations in plasma were determined using a commercially available ELISA assay (Neogen, Lexington, Ky.).

The results, summarized in Table 5, showed that AZT enhanced the in vivo antitumor effect of paclitaxel. First, treatment with the combination of paclitaxel and AZT resulted in a decrease in tumor size during a 10-day follow-up period, whereas animals in the control group, paclitaxel group, and AZT group showed an up to 4-fold increase in tumor size. The tumor size of the animals receiving the combination of paclitaxel and AZT at 10 days was significantly smaller than all other dose groups (p<0.001, ANOVA with repeated measures). Second, evaluation of the tumor morphology showed that the tumors of animals receiving the combination of paclitaxel and AZT had a 2- to 4-fold higher density of apoptotic cells, and a 2.6- to 4-fold lower density of non-apoptotic cells than all other dose groups. Third, survival analysis (i.e., Kaplan Meier analysis) showed that the median time to reach moribundity increased from 21-26 days for the control group and the AZT group to 42 days for the paclitaxel group and 49 days for the combination group. The paclitaxel group did not show tumor-free survivors whereas the combination group showed 2 tumor-free survivors (2 of 12, 16%). Survival of the group receiving the combination of paclitaxel and AZT was statistically longer than all other groups (p<0.01 by log rank test).

Treatments with single agents (either paclitaxel or AZT) produced minimal toxicity, with no toxicity-related death and minimal body weight loss compared to the pretreatment weight (<3%). The addition of AZT to paclitaxel did not enhance the body weight loss, indicating that AZT did not enhance the host toxicity of paclitaxel. The concentration of AZT in plasma, determined by an ELISA assay, was 9.6 nM. This concentration is well below the concentration required for inhibition of telomerase (2 μM in FaDu cells, see Table 1), or induction of cytotoxicity (20 μM in FaDu cells; Mo et al, Cancer Res. 63:579-585, 2003). Hence the result of this study is surprising.

TABLE 5
Enhancement of antitumor effect of paclitaxel by AZT

| | | | | End-of- |
|---|---|---|---|---|
| | | | | experiment |
| | Number of | Number of | | body weight, |
| | nonapoptotic | apoptotic | % Apoptotic | % of |
| | cells per | cells per | cells | pretreatment |
| Treatment (n) | 400x field | 400x field | per tumor | value |
| Saline control | 235 ± 39 | 31 ± 7 | 12 ± 2% | 106 ± 3 |
| (11) | | | | |
| AZT (10) | 249 ± 36 | 28 ± 7 | 10 ± 3% | 105 ± 6 |
| Paclitaxel (12) | 168 ± 53^{a} | 66 ± 34^{a} | 30 ± 17%^{a} | 97 ± 6^{a} |
| Paclitaxel + | 64 ± 68^{b} | 129 ± 36^{b} | 72 ± 26%^{b} | 99 ± 4^{a} |
| AZT (12) | | | | |

^{a}p < 0.05 compared to the control and AZT groups.
^{b}p < 0.05 compared to all other groups.
^{c}Data represents Mean ± SD of four independent sets of experiments. Cell density and apoptosis level were determined using image analysis at 4 randomized continuous tumor areas per tumor.

Conclusion. The results indicate that treatment with very low AZT doses, yielding nanomolar AZT concentrations in the plasma enhances the antitumor activity of chemotherapy.

### EXAMPLE 11

### Administration of Telomerase-Inhibiting Amounts of AZT Causes Tumor Size Reduction in Animals

Tumor size reduction after AZT in animals. The effect of repeated administration of single agent AZT on tumor size was determined in immunosuppressed mice implanted with FaDu tumors.

Treatment Protocol. Immunosuppressed mice were injected subcutaneously in both flanks with 10⁶ FaDu cells per flank. AZT was administered by continuous subcutaneous infusion using an Alzet® osmotic minipump at a rate of 5 μg/mouse/day. This AZT dose resulted in steady state plasma concentrations of about 10 ng/ml. AZT administration was started 14 days after inoculation, and lasted 14 days. The control animals were treated identically, except the AZT solution for injection was replaced by a physiologic saline solution. Tumor sizes were observed twice-weekly.

Effect of AZT treatment of tumor growth. All 16 animals implanted with tumor cells showed measurable tumors prior to AZT treatment. In two animals, the tumors stopped growing upon AZT administration, and subsequently declined in size. The tumors were not palpable on day 38, and completely disappeared on day 59, as confirmed by necropsy examination. For these two animals, the average initial tumor size was 35 mm³ (range, 18-80 mm³), which was similar to the tumor sizes in the remainder of the animals.

Conclusion. Long-term treatment with low dose of single agent AZT, a telomerase inhibitor, yielding nanomolar concentrations in the plasma, can cause complete disappearance of the tumor.

### EXAMPLE 12

### Telomerase Inhibition Combined With Tumor Size-Reducing Therapy is Expected to Improve Treatment Outcome

This example describes the expected enhancement of the antitumor effect of a cytoreductive treatment when the cytoreductive treatment is combined with long-term treatment with a telomerase inhibitor. The treatment with the telomerase inhibitor can start prior to, concurrently with, or after completion of the cytoreductive treatment.

Effect of long-term treatment with a telomerase inhibitor in combination with a cytoreductive treatment. In order for telomerase inhibitors to be effective to inhibit cell proliferation, or induce apoptosis and cell senescence, the tumor burden in the host must be small so that the tumor burden does not reach the lethal level before the telomerase inhibitor can erode the telomere length to below the critical level for inhibiting proliferation and for inducing apoptosis and cell senescence. This can be accomplished by using cytoreductive treatments. Hence, if a form of cytoreductive treatment is used in combination with long-term inhibition of telomerase activity, the expectation is that the telomere lengths in the tumor cells will decrease over time, and that, eventually, apoptosis or cell senescence will be induced.

Treatment Protocol Patients, presenting with a cancer for which the best treatment option is a form of cytoreductive therapy, would be treated with the cytoreductive treatment of choice. The cytoreductive treatment selected for an individual patient would depend on the patient's cancer, health status, age, previous treatment history, and other factors usually considered in the selection of a treatment protocol. Upon completion of the cytoreductive treatment regimen, and if the patient is not considered cured, or recurrence of the disease is considered likely, the patient would receive continuing treatment with a telomerase inhibitor, at a dose level that is sufficient to induce inhibition of telomerase. The duration of the continuing treatment would be protracted, lasting at least two weeks, but preferably longer than two months, or more preferably longer than six months, or more preferably until the patient is considered cured. An alternative treatment protocol, where the telomerase inhibitor treatment is initiated prior to, or concurrently with the cytoreductive treatment, and continued after completion of the cytoreductive treatment regimen, would be advantageous, as the tumor would be exposed to the telomerase inhibiting effect for a longer period of time.

Conclusion. The expectation is that combining telomerase-inhibiting treatment with conventional cytoreductive treatment approaches would enable the telomerase inhibiting treatment to become effective, and would solve the long-standing problem of defining a method of advantageously using telomerase inhibition to treat patients afflicted with a cancer.

### EXAMPLE 13

### Local Administration of Suramin Resulted in telomerase-Inhibitory Concentrations in the Intended Target Organ But Not in the Plasma

This example describes regional administration of a telomerase inhibitor, to achieve telomerase inhibitory concentrations in the targeted tissue or organ, while presenting low and not effective telomerase inhibitory concentrations in the plasma.

Suramin bladder wall and systemic concentrations after regional delivery. A solution of suramin was instilled into the bladder cavity of dogs, and bladder wall concentrations as a function distance from the bladder cavity, as well as systemic plasma concentrations, were studied.

Treatment protocol Five beagle dogs, weighing 9.0±0.4 kg, were used. The animals were cannulated in a cephalic vein for administration of anesthetics, and in a jugular vein for blood sampling. A urethral catheter was inserted for dose instillation and sampling of the bladder contents. Animals were given intravesical doses of suramin (20 ml containing 6 mg/ml suramin) in water. Concentrations of the bladder contents and systemic plasma were sampled at frequent intervals for 120 minutes, at which time the bladder tissue was harvested and the animal sacrificed. Tissue sections of approximately 2 cm×2 cm surface area were cut from the bladder wall, and rapidly frozen on a flat stainless steel plate cooled on dry ice. The outer edges of the tissue samples were trimmed to avoid contamination with instillation fluid, and the frozen tissues cut into 40 μm sections parallel to the urothelial surface. Suramin concentrations in the tissue layers, plasma and bladder contents were determined by HPLC analysis.

Results. For animals receiving intravesical suramin at 6 mg/ml, concentrations in the bladder contents declined from 6 mg/ml at 0 minutes to 3.6 mg/ml at 120 minutes. Plasma concentrations were less than 0.1 μg/ml at all times. The concentrations in the bladder wall tissue declined from the urethral surface (0 mm) to the serosal surface (4-5 mm). The tissue concentration at 0 mm was approximately 80 μg/g, and showed a log-linear decline until approximately 2 mm, where a concentration of approximately 3 μg/g was reached. The concentration in the remainder of the bladder wall showed little variation with depth, and was approximately 3 μg/g.

Conclusion. These results show that regional drug administration in an organ can provide telomerase-inhibitory concentrations in the intended organ or tissue, while systemic plasma concentrations are many-fold lower and not necessarily telomerase inhibitory.

### EXAMPLE 14

### Cell Culture Systems

Cell culture assay experiments can be performed in the human prostate PC3 tumor cells, the human breast MCF7 cells, or the human pharynx FaDu cells. If the requirements of the invention are met in any of the three cell cells, the choice and the dosage of the telomerase inhibitor is suitable for use in the invention. Preferably the PC3 cells are used.

Human prostate PC3 tumor cells, breast MCF7 cells, or pharynx FaDu cells can be obtained from the American Type Culture Collection. The doubling time of all three cell lines is approximately 24-hour. All three cell lines should be cultured as monolayers in a humidified environment containing 5% CO₂ and 95% air, at 37° C. PC3 cells should be maintained in RPMI 1640 medium, MCF7 cells in either RPMI 1640 or Minimal Essential Medium (MEM), and FaDu cells in MEM. All culture media should be supplemented with 9% heat-inactivated fetal bovine serum, 2 mM 1-glutamine, 0.1% 10 mM non-essential amino acids, 90 μg/ml gentamicin, and 90 μg/ml cefotaxime sodium. Cells are harvested from subconfluent cultures using trypsin and resuspended in fresh medium before plating. Cells with >90% viability, as determined by trypan blue exclusion, are used to evaluate the cytotoxicity of a telomerase inhibitor, e.g., suramin. Cells are plated in 96 well microtiter plates at a density such that confluence would not be achieved at the end of the drug treatment period. Cells are allowed to attach to the plate surface by growing in drug-free medium for 20 to 24 hr. Afterward, cells are incubated with the FGF antagonist (one example used 0.2 ml of suramin)-containing culture medium, at concentrations spanning at least 4 log scales. The drug effect should be measured as inhibition of BrdU incorporation, e.g., according to the Cell Proliferation ELISA BrdU (Boehringer Mannheim).

Conclusion

While the invention has been described with reference to and illustrated by a variety of embodiments, those skilled in the art will understand that various changes may be made and equivalents may be substituted for elements thereof without departing from the scope of the invention. In addition, many modifications may be made to adapt a particular situation or material to the teachings of the invention without departing from the essential scope thereof. As those skilled in the art will recognize, or be able to ascertain, using no more than routine experimentation, many equivalents to specific embodiments of the invention described specifically herein. Such equivalents are encompassed in the scope of the following claims. Therefore, it is intended that the invention not be limited to the particular embodiment disclosed as the best mode contemplated for carrying out this invention, but that the invention will include all embodiments falling within the scope of the appended claims. In this application all units are in the metric system and all amounts and percentages are by weight, unless otherwise expressly indicated. Also, all citations referred herein are expressly incorporated herein by reference.

### REFERENCES CITED

U.S. Pat. No. 5,116,823
U.S. Pat. No. 5,489,508.
U.S. Pat. No. 5,597,830.
U.S. Pat. No. 5,656,638.
U.S. Pat. No. 5,760,062.
U.S. Pat. No. 5,767,110.
U.S. Pat. No. 5,767,278.
U.S. Pat. No. 5,770,613.
U.S. Pat. No. 5,863,936.
U.S. Pat. No. 6,452,014.
WO patent 93 23572.
WO patent 97 38013 A
Ahmann, F. R., J. Schwartz, R. Dorr, and S. Salmon, Suramin in hormone resistant metastatic prostatic cancer: Significant anticancer activity but unanticipated toxicity., Proc. Am. Soc. Clin. Oncol., 10, 178, 1991.
Armand, J. P., M. Bonnay, D. Gandia, E. Cvitkovic, F. De Braud, F. Bertheault, J. P. Droz, P. Carde, M. Schlumberger, and D. Fourcault, Phase I-II suramin (SRM) study in advanced cancer patients. (abstract), Proc. Am. Assoc. Cancer Res., 32, 175, 1991.
Armitage, J. O., D. D. Weisenburger, M. Hutchins, D. F. Moravec, M. Dowling, S. Sorensen, J. Mailliard, J. Okerbloom, P. S. Johnson, D. Howe, and et al., Chemotherapy for diffuse large-cell lymphoma—rapidly responding patients have more durable remissions., J Clin. Oncol., 4, 160-164, 1986.
Burger, A. M., J. A. Double, and D. R. Newell, Inhibition of telomerase activity by cisplatin in human testicular cancer cells., Eur J Cancer, 33, 638-644, 1997.
Clark, J., W. Sikov, F. Cumings, M. Browne, W. Akerley, H. Wanebo, A. Weitberg, T. Kennedy, B. Cole, J. Bigley, J. Beitz, and J. Darnowski, Phase II study of 5-fluoruracil leucovorin and azidothymidine in patients with metastatic colorectal cancer., J Cancer Res. Clin. Oncol., 122, 554-558, 1996.
DeClercq, E., Suramin: A potent inhibitor of the reverse transcriptase of RNA tumor viruses., Cancer Letters, 8, 9-22,1979.
Dimri, C. P., Lee, X., Basile, G., Acosta, M., Scott, G., Roskelley, C., Medrano, E. E., Linskens, M., Rubeli, I., Pereira-Smith, O., and Peacocke, M. A biomarker that identifies senescent human cells in culture and in aging skin in vivo. Proc. Natl. Acad. USA., 92: 9363-9367, 1995.
Dreicer, R., D. C. Smith, R. D. Williams, and W. A. See, Phase II trial of suramin in patients with metastatic renal cell carcinoma., Invest. New Drugs, 17, 183-189, 1999.
Eisenberger, M. A. and L. M. Reyno, Suramin, Cancer Treat Rev., 20, 259-273, 1994.
Falcone, A., A. Antonuzzo, R. Danesi, G. Allegrini, L. Monica, E. Pfanner, G. Masi, S. Ricci, M. Del Tacca, and P. F. Conte, Suramin in combination with weekly epirubicin for patients with advanced hormone-refractory prostate carcinoma., Cancer, 86, 470-476, 1999.
Falcone, A., E. Pfanner, I. Brunetti, G. Allegrini, M. Lencioni, C. Galli, G. Masi, R. Danesi, A. Antonuzzo, M. Del Tacca, and P. F. Conte, Suramin in combination with 5-fluorouracil (5-FU) and leucovorin (LV) in metastatic colorectal cancer patients resistant to 5-FU+LV-based chemotherapy., Tumori, 84, 666-668, 1998.
Feng, J., W. D. Funk, S. S. Wang, S. L. Weinrich, A. A. Avilion, C. P. Chiu, R. R. Adams, E. Chang, R. C. Allsopp, J. Yu, and et. al., The RNA component of human telomerase., Science, 269, 1236-1241, 1995.
Fotes, P. A., J. C. Ellory, and V. L. Lew, Suramin: a potent ATPase inhibitor which acts on the inside surface of the sodium pump., Biochim Biophys Acta., 38, 262-272, 1973.
Funayama, Y., K. Nishio, Y. Takeda, N. Kubota, T. Ohira, T. Ohmori, S. Ohta, H. Ogasawara, S. Hasegawa, and N. Saijo, Suramin inhibits the phosphorylation and catalytic activity of DNA topoisomerase 11 in human lung cancer cells., Anticancer Res., 13, 1981-1988, 1993.
Gan, Y., K. J. Engelke, C. A. Brown, and J. L. Au, Telomere amount and length assay., Pharm. Res., 18, 1655-1659, 2001.
Gan, Y., J. Lu, A. Johnson, M. G. Wientjes, D. E. Schuller, and J. L. S. Au, A quantitative assay of telomerase activity., Pharm. Res., 18, 488-493, 2001.
Gan, Y., Y. Mo, J. Johnston, J. Lu, M. G. Wientjes, and J. L. S. Au, Telomere maintenance in telomerase-positive human ovarian SKOV-3 cells cannot be retarded by complete inhibition of telomerase., FEBS Lett., 527, 10-14, 2002.
Garrett, J. S., S. R. Coughlin, H. L. Niman, P. M. Tremble, G. M. Giels, and L. T. Williams, Blockade of autocrine stimulation in simian sarcoma virus transformed cells reverses down-regulation of platelet-derived growth factor receptors., Proc. Natl. Acad. Sci. USA, 81, 7466-7470, 1984.
Gibaldi, M., et at., Pharmacokinetics., Marcel Dekker, Inc., New York, 1982.
Goldstein, S., Replicative senescence: the human fibroblast comes of age., Science, 249, 1129-1133, 1990.
Grazioli, L., R. Alzani, M. Coimei, M. Mariani, A. Restivo, E. Cozzi, and F. Marcucci, Inhibitory effect of suramin on receptor binding and cytotoxic activity of tumor necrosis factor., Int J Immunopharmacol, 14, 637-642, 1992.
Hawking, F., Suramin: with special reference to onchocerciasis., Adv. Pharmacol. Chemother., 5, 289-322, 1978.
Henderson, S., R. Allsopp, D. Spector, S. S. Wang, and C. Harley, In situ analysis of changes in telomere size during replicative aging and cell transformation., J. Cell Biol., 134, 1-12, 1996.
Hensey, C. E., D. Boscoboinik, and A. Azzi, Suramin, an anti-cancer drug, inhibits protein kinase C and induces differentiation in neuroblastoma cell clone NB2A., FASEB, 258,156-158, 1989.
Hiyama, E., L. Gollahon, T. Kataoka, K. Kuroi, T. Yokoyama, A. F. Gazdar, K. Hiyama, M. S. Piatzszek, and J. W. Shay, Telomerase activity in human breast tumors., J Natl Cancer Inst, 88, 116-122, 1996.
Hosang, M., Suramin binds to platelet-derived growth factor and inhibits its biological activity., J. Cell. Biochem., 29, 265-273, 1985.
Huang, A. and J. A. Wright, Fibroblast growth factor mediated alterations in drug resistance, and evidence of gene amplification., Oncogene, 9, 491-499, 1994.
Hussain, M., E. I. Fisher, D. P. Petrylak, J. O'Connor, D. P. Wood, E. J. Small, M. A. Eisenberger, and E. D. Crawford, Androgen deprivation and four courses of fixed-schedule suramin treatment in patients with newly diagnosed metastatic prostate cancer: A southwest oncology group study., J of Clin. Oncol., 18, 1043-1049, 2000.
Jodrell, D. I., L. M. Reyno, R. Sridhara, M. A. Eisenberger, K. H. Tkaczuk, E. G. Zuhowski, V. J. Sinibaldi, M. J. Novak, and M. J. Egorin, Suramin: development of a population pharmacokinetic model and its use with intermittent short infusions to control plasma drug concentration in patients with prostate cancer, J Clin. Oncol., 12,166-175, 1994.
Kassack, M. and P. Nickel, Rapid, highly sensitive gradient narrow-bore high-performance liquid chromatographic determination of suramin and its analogues., J Chromatogr. B Biomed. Appl., 686, 275-284, 1996.
Kondo, S., Y. Kondo, G. Li, R. H. Silverman, and J. K. Cowell, Targeted therapy of human malignant glioma in a mouse model by 2-5A antisense directed against telomerase RNA., Oncogene, 16, 3323-3330, 1998.
Kuh, H. J., S. H. Jang, M. G. Wientjes, and J. L. S. Au, Determinants of paclitaxel penetration and accumulation in human solid tumor., J. Pharmacol. Exp. Therap., 290, 871-880, 1999.
Lingner, J., J. P. Cooper, and T. R. Cech, Telomerase and DNA end replication: no longer a lagging strand problem?, Science, 269, 1533-1534, 1995.
Lush, R. M., W. D. Figg, J. M. Pluda, R. Bitton, D. Headlee, D. Kohler, O. Sartor, E. Reed, and M. R. Cooper, A phase I study of pentosan polysulfate sodium in patients with advanced malignancies., Ann Oncol., 7, 939-944, 1996.
Magabhushan, M., C. M. Miller, T. P. Pretlow, J. M. Giaconia, N. L. Edgehouse, S. Schwartz, H.-J. Kung, R. W. de Vere White, P. H. Gumerlock, M. I. Resnick, S. B. Amini, and T. G. Pretlow, CWR22 The first human prostate cancer xenograft with strongly androgen-dependent and relapsed strains both in vivo and in soft agar., Cancer Res., 56, 3042-3046, 1996.
Manetti, F., V. Cappello, M. Botta, F. Corelli, N. Mongelli, G. Biasoli, A. Lombardi Borgia, and M. Ciomei, Synthesis and binding mode of heterocyclic analogues of suramin inhibiting the human basic fibroblast growth factor., Bioorganic & Med. Chem., 6, 947-958, 1998.
Martin, G. M., C. A. Sprague, and C. J. Epstein, Replicative life-span of cultivated human cells. Effects of donor's age, tissue, and genotype., Lab. Invest., 23, 86-92, 1979.
Marshall J L Wellstein A Rae J DeLap R J Phipps K Hanfelt J Yunmbam M K Sun J X Duchin K L Hawkins M J. Phase I trial of orally administered pentosan polysulfate in patients with advanced cancer. Clin. Cancer Res., 3:2347-2354, 1997.
McLeskey, S. W., L. Zhang, B. J. Trock, S. Kharbanda, U. Liu, M. M. Gottardis, M. E. Lippman, and F. G. Kern, Effects of AGM-1470 and pentosan polysulfate on tumorigenicity and metastasis of FGF-mediated MCF-7 cells., Br. J. Cancer, 73, 1053-1062, 1996.
Melana, S. M., J. F. Holland, and B. G. Pogo, Inhibition of cell growth and telomerase activity of breast cancer cells in vitro by 3′-azido-3′-deoxythymidine., Clin. Cancer Res., 4, 693-696, 1998.
Miglietta, L., L. Canobbio, C. Granetto, M. Vannozzi, M. Esposito, and F. Boccardo, Suramin/epidoxorubicin association in hormone-refractory prostate cancer: preliminary results of a pilot phase II study., J Cancer Res. Clin. Oncol., 123, 407-410, 1997.
Mills, G. B., N. Zhang, C. May, M. Hill, and A. Chung, Suramin prevents binding of Interleukin 2 to its cell surface receptor: A possible mechanism for immunosuppression., Cancer Res., 50, 3036-3042, 1990.
Mirza, M. R., E. Jakobsen, P. Pfeiffer, B. Lindebjerg-Clasen, J. Bergh, and C. Rose, Suramin in non-small cell lung cancer and advanced breast cancer. Two parallel phase II studies., Acta Oncologica, 36, 171-174, 1997.
Mo, Y., Gan, Y., Song, S. H., Johnston, J., Xiao, X., Wientjes, M. G., Au, J. L.-S. Simultaneous targeting of telomere and telomerase as a cancer therapeutic approach. Cancer Res., 63:579-585, 2003.
Motzer, R. J., E. Dmitrovsky, W. H. Miller, W. P. Tong, D. F. Bajorin, H. I. Scher, and G. J. Bosl, Suramin for Germ Cell Tumors. In vitro growth inhibition and results of a Phase II trial., Cancer, 72, 3313-3317, 1993.
Multani, A. S., V. L. Hopwood, and S. Pathak, A modified fluorescence in situ hybridization (FISH) technique., Anticancer Res., 16, 3435-3437, 1996.
Myers, C. E., R. LaRocca, C. Stein, M. Cooper, N. Dawson, P. Choyke, M. Linehan, and M. Urich, Treatment of hormonally refractory prostate cancer with suramin., Proc. Am. Soc. Clin. Oncol., 9, 113, 1990.
Nguyen, M., J. E. Lehr, and K. J. Pienta, Pentosan inhibits angiogenesis in vitro and suppresses prostate tumor growth in vivo., Anticancer Res., 13, 2143-2148, 1993.
Norton, J. C., M. A. Piatyszek, W. E. Wright, J. W. Shay, and D. R. Corey, Inhibition of human telomerase activity by peptide nucleic acids., Nat Biotechnol, 14, 615-619, 1996.
Ono, K., H. Nakane, and M. Fukushima, Differential inhibition of various deoxyribonucleic and ribonucleic acid polymerases by suramin., Eur. J. Biochem., 172, 349-353, 1998.
Parker, B. W., S. M. Swain, G. Zugmaier, R. L. DeLap, M. E. Lippman, and A. Wellstein, Detectable inhibition of heparin-binding growth factor activity in sera from patients treated with pentosan polysulfate., J. Natl. Cancer Inst., 85, 1068-1073, 1993.
Pesenti, E., F. Sola, N. Mongelli, M. Grandi, and F. Spreafico, Suramin prevents neovascularisation and tumour growth through blocking of basic fibroblast growth factor activity., Br. J. Cancer, 66, 367-372, 1992.
Pollak, M. and M. Richard, Suramin blockade of insulin like growth factor I-Stimulated proliferation of human osteosarcoma cells., J. Natl. Cancer Inst., 82, 1349-1352, 1990.
Pollak, M. and M. Richard, Suramin interferes with the binding of insulin-like growth factor I (IGF-I) to human osteosarcoma cells and blocks IGF-I stimulated proliferation., Proc. Am. Soc. Clin. Oncol., 9, 54, 1990.
Rapoport, B. L., G. Falkson, J. I. Raats, M. de Wet, B. P. Lotz, and H. C. Potgieter, Suramin in combination with mitomycin C in hormone-resistant prostate cancer. A phase II clinical study., Ann. Oncol., 4, 567-573, 1993.
Rosen, P. J., E. F. Mendoza, E. M. Landaw, B. Mondino, M. D. Graves, J. H. McBride, P. Turcillo, J. deKernion, and A. Belidegrun, Suramin in hormone-refractory metastatic prostate cancer: A drug with limited efficacy., J. Clin. Oncol., 14, 1626-1636, 1996.
Skehan, P., R. Storeng, D. Scudiero, A. Monks, J. McMahon, D. Vistica, J. T. Warren, H. Bokesch, S. Kenney, and M. R. Boyd, New colorimetric cytotoxicity assay for anticancer-drug screening., J. Natl. Cancer Inst., 82, 1107-1112, 1990.
Song, S., M. G. Wientjes, Y. Gan, and J. L. S. Au, Fibroblast growth factors: An epigenetic mechanism of broad spectrum resistance to anticancer drugs., Proc. Natl. Acad. Sci. USA, 97, 8658-8663, 2000.
Stein, C. A., Suramin: A novel antineoplastic agent with multiple potential mechanisms of action., Cancer Res., 54, 2239-2248, 1993.
Strahl, C. and E. H. Blackburn, The effects of nucleoside analogs on telomerase and telomeres in Tetrahymena., Nucleic Acid Res, 22, 893-900, 1994.
Strahl, C. and E. H. Blackburn, Effects of reverse transcriptase inhibitors on telomere length and telomerase activity in two immortalized human cell lines. Mol. Cell Biol., 16, 53-65, 1996.
Swain, S. M., B. W. Parker, A. Wellstein, M. E. Lippman, C. Steakley, and R. L. DeLap, Phase I trial of pentosan ploysulfate., Invest. New Drugs, 13, 55-62, 1995.
Takano, S., S. Gately, M. E. Neville, W. F. Herblin, J. L. Gross, H. Engelhard, M. Perricone, K. Eidsvoog, and S. Brem, Suramin, an anticancer and angiosuppressive agent, inhibits endothelial cell binding of basic fibroblast growth factor, migration, proliferation, and induction of urokinase-type plasminogen activator., Cancer Res., 54, 2654-2660, 1994.
Villalona-Calero, M., Wientjes, M. G., Otterson, G. A. Kanter, S. Young, D., Murgo, A. J., Fischer, B., DeHoff, Ca., Chen, D., Yeh, T.-K., Song, S-H., Grever, M., Au, J. L.-S. Phase I study of low dose suramin as a chemosensitizer in patients with advanced non-small cell lung cancer. Clin. Cancer Res., 9:3303-3311, 2003.
Villalona-Calero, M., Otterson, G. A., Wientjes, M. G., Murgo, A. J., Jensen, R., Yeh, T.-K., Chen, D., Song, S-H., Grever, M., Au, J. Phase II evaluation of low dose suramin as a modulator of paclitaxel/carboplatin in non-small cell lung cancer (NSCLC) patients. IASLC Meeting, Vancouver, August, 2003
Wade, T. P., A. Kasid, C. A. Stein, R. LaRocca, E. R. Sargent, L. G. Gomella, C. E. Myers, and W. M. Linehan, Suramin interference with transforming growth factor-inhibition of human renal cell carcinoma in culture., J. Surg. Res., 53, 195-198, 1992.
Waltenberger, J., U. Mayr, H. Frank, and V. Hombach, Suramin is a potent inhibitor of vascular endothelial growth factor. A contribution to the molecular basis of its antiangiogenic action., J. Mol. Cell Cardiol., 28, 1523-1529, 1996.
Wellstein, A. and F. Czubayko, Inhibition of fibroblast growth factors., Breast Cancer Res. Treat., 38, 109-119, 1996.
Wellstein, A., G. Zugmaier, J. A. Califano, S. Paik, and M. E. Lippman, Tumor growth dependent on Kaposi's sarcoma-derived fibroblast growth factor inhibited by pentosan polysulfate., J. Natl. Cancer Inst., 83, 716-720, 1991.
Zugmaier, et al., Ann. NY Acad. Sciences, 886:243-248,1999.
Physician Desk Reference, 2003. http://pdrel.thomsonhc.com.

1. A method of improving therapeutic outcome of treating a cancer patient having a tumor, which comprises the steps of:
(a) determining the tumor burden of the cancer patient;
(b) subjecting the tumor to cytoreduction if the determined tumor burden is sufficient to preclude an administered telomerase inhibitor from being present in the cancer patient for a duration of at least several cycles of tumor cell proliferation; and
(c) administering to the cancer patient a telomerase-inhibiting amount of telomerase inhibitor comprising suramin or a pharmaceutically acceptable salt of suramin, wherein the tumor burden is such that the tumor is exposed to the suramin for a duration of time of at least several cycles of tumor cell proliferation for improving the therapeutic outcome of treating said cancer patient.

2. The method of claim 1, wherein said cancer patient is a mammal.

3. The method of claim 1, wherein said telomerase inhibitor amount ranges from between about 0.0001 to about 100 mg per kilogram of weight of said cancer patient.

4. The method of claim 3, wherein said telomerase-inhibiting amount ranges from between about 0.01 to about 10 mg per kilogram of weight of said cancer patient.

5. The method of claim 4, wherein said telomerase-inhibiting amount ranges from between about 0.1 to about 4 mg per kilogram of weight of said cancer patient.

6. The method of claim 1, wherein said duration of time ranges from about one day to about 365 days.

7. The methods of claim 6, wherein said duration of time is for a maintenance period of time.

8. The method of claim 1, wherein said telomerase inhibitor comprises suramin administered in an amount that results in a plasma concentration in said mammal of between about 0.001 and 100 μg/ml.

9. The method of claim 8, wherein suramin is administered in an amount that results in a plasma concentration in said mammal of between about 10 and 70 μg/ml.

10. The method of claim 1, wherein said telomerase inhibitor comprises suramin and said mammal is exposed to less than about 7,840 μM-day of suramin in plasma over 112 days.

11. The method of claim 1, wherein said telomerase inhibitor comprises suramin and said mammal is exposed to no more than about 800 μM of suramin over 96 hours.

12. The method of claim 1, wherein cytoreductive treatment is one or more of surgical excision of tumor, radiation therapy, chemotherapy, or photodynamic therapy.

13. The method of claim 1, wherein said cancer patient is afflicted with one or more of fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, colon carcinoma, rectal cancer, pancreatic cancer, breast cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, Kaposi's sarcoma, acute promyeloid leukemia (APML), acute myelogenous leukemia (AML), chronic myelogenous leukemia (CML), acute lymphoblastic leukemia, chronic lymphocytic leukemia (CLL), prolymphocytic leukemia (PLL), hairy cell leukemia (HLL), Waldenstrom's macroglobulinemia (WM), non-Hodgkin's lymphoma, peripheral T-cell lymphomas, adult T-cell leukemia/lymphoma (ATL), cutaneous T-cell lymphoma (CTCL), large granular lymphocytic leukemia (LGF), erythroleukemias, lymphomas, Hodgkin's disease, embryonic carcinomas, or embryonic teratomas.

14. The method of claim 1, wherein said several cycles of tumor cell proliferation ranges from about 3 to 26 cell doublings of cells of said tumor.

15. The method of claim 1, wherein said administering in step (c) commences one or more of: prior to any cytoreduction in step (b), concurrent with any cytoreduction in step (b), or after any cytoreduction in step (b).

16. The method of claim 1, wherein the administration of the telomerase inhibitory agent is regionally and wherein tissue concentrations of the inhibitory agent are sufficient to inhibit telomerase activity in tumor cells.

17. The method of claim 16, where the treatment does not result in telomerase-inhibitory concentrations in one or more of plasma or other organs that are not the targets of the treatment.

18. The method of claim 1, wherein said tumor comprises a telomerase-dependent tumor.

19. The method of claim 1, wherein said telomerase inhibitor is administered to said cancer patient by one or more of the following routes of administration:
subcutaneously, intravenously, intramuscularly, intraperitoneally, intradermally, intravesically, intrathecally, orally, nasally, intrapulmonary by inhalation, rectally, topically, locally, regionally, or transdermally.

20. The method of claim 1, wherein said telomerase inhibitor is in a timed-release formulation.

21. The method of claim 1, wherein said telomerase inhibitor is formulated into one or more of a solid or liquid for administration.

22. The method of claim 1, wherein said telomerase inhibitor is added to an adjuvant.

23. The method of claim 22, wherein said adjuvant is one or more of a preservative, a wetting agent, an emulsifying agent, or a dispersing agent.

24. The method of claim 1, wherein said telomerase inhibitor is in the form of a microparticle having an average particle size of between about 10 nm and 300 μ.

25. The method of claim 24, wherein said microparticle has an average particle size of between about 10 nm and 300 nm.

26. A method of enhancing the therapeutic outcome of treating a cancer patient having a tumor, where the treatment comprises:
administering to the cancer patient a telomerase-inhibiting amount of a telomerase inhibitor comprising suramin.

27. The method of claim 26, wherein the administering of a telomerase inhibitor is conducted in such a manner that the tumor is exposed to the telomerase inhibitor for a duration of at least several cycles of tumor cell proliferation.

28. The method of claim 26, wherein said telomerase inhibitor has an IC₅₀ of less than about 10 μM.

29. A kit comprising:
(a) a telomerase inhibitory agent comprising suramin or a pharmaceutically acceptable salt of suramin in a pharmaceutically acceptable carrier,
(b) a container; and
(c) directions for using said telomerase inhibitory agent for one or more of inhibiting or reducing aberrant growth associated with a tumor.

30. A method of treating a patient with minimal neoplastic disease not requiring cytoreduction, which comprises the steps of:
(a) first administering to the patient a telomerase inhibitor comprising suramin or a pharmaceutically acceptable salt of suramin in a amount that is effective to inhibit telomerase activity, and
(b) second, at the time that recurrence of the tumor is recognized, administering to the patient a cytotoxic chemotherapy regimen that includes a telomerase inhibitor comprising suramin, where the addition of the telomerase inhibitor to the chemotherapy regimen enhances the efficacy of the cytotoxic chemotherapy regimen.

31. The method of claim 30, wherein said telomerase inhibitor is suramin administered at a dose level where suramin inhibits telomerase activity, but does not cause a substantial cytotoxic effect.

32. The method of claim 31, wherein said patient is a mammal.

33. The method of claim 31, wherein the administration of the telomerase inhibitory agent is regionally and wherein the tissue concentrations of the inhibitory agent are sufficient to inhibit telomerase activity in tumor cells.

34. The method of claim 33, where the treatment does not result in telomerase-inhibitory concentrations in plasma or other organs that are not the targets of the treatment.

35. A method of treating a patient, which comprises the steps of:
(a) identifying a patient, which is one or more of:
(1) about to have a cancer, or
(2) harboring a cancer that is too small to be detected by conventional means comprising one or more of palpation, detection of blood in urine, detection of blood in a stool, or the use of imaging modalities comprising one or more of X-ray, CAT scan, PET scan, or ultrasound imaging; and
(b) administering a telomerase inhibitory agent comprising suramin or a pharmaceutically acceptable salt of suramin to the patient, such that one or more of treatment of the cancer or prevention of cancer development is achieved.

36. The method of claim 35, wherein said telomerase inhibitor is suramin administered at a dose level where suramin inhibits telomerase activity, but does not cause substantial cytotoxic effects.

37. The method of claim 34, wherein said patient is a mammal.

38. The method of claim 37, wherein the administration of the telomerase inhibitory agent is regionally and wherein the tissue concentrations of the inhibitory agent are sufficient to inhibit telomerase activity in tumor cells.

39. The method of claim 38, where the treatment does not result in telomerase-inhibitory concentrations in plasma or other organs that are not the targets of the treatment.

40. A method of treating a cancer patient, which comprises:
administering to the cancer patient a telomerase inhibitory amount of suramin or a pharmaceutically acceptable salt of suramin during and after completion of a cytoreductive treatment, where the addition the telomerase inhibitor to the cytoreductive treatment improves the treatment outcome of the patient.

41. A method of treating a cancer patient, which comprises:
administering to said patient 3′-azido-deoxythymidine (AZT), such that treatment of the cancer is achieved, wherein plasma concentrations of AZT in the patient are below the concentrations used for the treatment of HIV infection.

42. The method of claim 41, wherein the patient has a tumor and the AZT is administered to the patient one or more of: concurrent with or after a surgical cytoreductive treatment of the tumor has been performed on the patient.

43. The method of claim 42, wherein the patient has a tumor and the AZT is administered to the patient one or more of: prior to, concurrent with, or after the patient is subjected to a nonsurgical cytoreductive treatment of the tumor.

44. The method of claim 42, wherein plasma concentrations of AZT in the plasma of the patient are in the nanomolar range.

45. A method of enhancing therapeutic outcome of treating patient having telomerase-mediated disease, which comprises the steps of:
administering to the patient a telomerase-inhibiting amount of a telomerase inhibitor comprising one or more of suramin or a pharmaceutically acceptable salt of suramin.

46. The method of claim 45, wherein said patient is a mammal.

47. A method for enhancing therapeutic outcome of treating a cancer patient having a tumor with a telomerase-inhibiting amount of a telomerase inhibitor, which comprises the steps of:
(a) obtaining a sample of said tumor;
(b) subjecting said tumor sample to terminal restriction fragment (TRF) analysis to determine the lengths of the terminal fragments containing the telomere DNA of the cells in the sample; and
(c) correlating the length of the telomere DNA with the length of time required to treat the cancer patient with a telomerase-inhibiting amount of a telomerase inhibitor being one or more of suramin, a pharmaceutically acceptable salt of suramin, pentosan polysulfate (PPS), a pharmaceutically acceptable salt of PPS, or hTR-antisense transfection.

48. The method of claim 47, wherein said patient is a mammal.

49. A method for determining the therapeutic efficacy of treating a cancer patient having a tumor with a telomerase-inhibiting amount of a telomerase inhibitor, which comprises the steps of:
(a) obtaining a sample of said tumor;
(b) subjecting said tumor sample to terminal restriction fragment (TRF) analysis to determine the lengths of the terminal fragments containing the telomere DNA of the cells in the sample; and
(c) correlating -the length of the telomere DNA with the course of said treating.

50. The method of claim 49, wherein said patient is a mammal.

51. A method of enhancing therapeutic outcome of treating patient having bladder interstitial cystitis, which comprises the steps of: locally administering to the patient an effective amount of one or more of suramin or a pharmaceutically acceptable salt of suramin.

52. A method of improving therapeutic outcome of treating a cancer patient having a tumor, which comprises the steps of:
(a) determining the tumor burden of the cancer patient;
(b) subjecting the tumor to cytoreduction if the determined tumor burden is sufficient to preclude an administered telomerase inhibitor from being present in the cancer patient for a duration of at least several cycles of tumor cell proliferation; and
(c) administering to the cancer patient a telomerase-inhibiting amount of telomerase inhibitor comprising one or more of pentosan polysulfate (PPS), a pharmaceutically acceptable salt of PPS, or hTR-antisense transfection of cells of said tumor, wherein the tumor burden is such that the tumor is exposed to the suramin for a duration of time of at least several cycles of tumor cell proliferation for improving the therapeutic outcome of treating said cancer patient.

53. The method of claim 52, wherein said telomerase inhibitor comprises PPS administered in an amount that results in a plasma concentration in said mammal of between about 0.001 and 1 μg/ml.
